(19) United States
(12) Patent Application Publication
(10) Pub. No.: US 2025/0270216 A1
(43) Pub. Date: Aug. 28, 2025

(54) ANTI-INFECTIVE AGENTS

(71) Applicant: UNIVERSITY OF DUNDEE, Dundee (GB)

(72) Inventors: Ian Hugh GILBERT, Dundee (GB); Beatriz BARAGAÑA RUIBAL, Dundee (GB); Nicola CALDWELL, Dundee (GB); Malcolm TAYLOR, Dundee (GB); Barbara FORTE, Dundee (GB); Mattia COCCO, Dundee (GB); Chimed JANSEN, Dundee (GB)

(21) Appl. No.: 18/859,690

(22) PCT Filed: Apr. 20, 2023
(86) PCT No.: PCT/GB2023/051034

§ 371 Date: Oct. 24, 2024

(30) Foreign Application Priority Data
Apr. 26, 2022 (GB) 2206076.8

(51) Int. Cl. C07D 487/04; A61K 31/519; A61P 33/02

(52) U.S. Cl. CPC C07D 487/04; A61K 31/519; A61P 33/02

(57) ABSTRACT

The invention relates to compounds or pharmaceutically acceptable salts thereof, compositions containing them, including combinations with at least one additional therapeutic agent, and their use in therapy, for example in the treatment of infectious diseases or in the treatment of diseases caused by Cryptosporidium.

### FIELD OF THE INVENTION

The invention relates to compounds or pharmaceutically acceptable salts thereof, compositions containing them, including combinations with at least one additional therapeutic agent, and their use in therapy, for example in the treatment of infectious diseases or in the treatment of diseases caused by Cryptosporidium.

### BACKGROUND

Cryptosporidiosis is a diarrhoeal disease caused by the parasite species Cryptosporidium. Currently, there are 27 recognized species of Cryptosporidium, including 20 species infecting humans with C. parvum or C. hominis being responsible for the majority of human infections, Int. J. For Parasitology 2015, 45, 367-373. Cryptosporidiosis was first identified as a cause of human infection in 1976, Gastroenterology, 1976, 70, 592-598. A more recent study investigating the cause and effect of diarrhoea in more than 22,000 children under the age of 5 years, recognized Cryptosporidium as the second most common cause of both diarrhoea and morbidity, after rotavirus, The Lancet, 2013, 382, 9888, 209-222.

Cryptosporidiosis is a major cause of diarrhoea in many parts of the world, particularly affecting children in sub-Saharan Africa and South Asia. The recent Global Enteric Multicenter Study (GEMS) estimates 2.9-4.7 million cases in children under 24 months of age in sub-Saharan Africa and the Indian sub-continent, leading to more than 200,000 deaths annually. Cryptosporidiosis is also associated with malnutrition and stunted growth. It is one of the leading causes of death due to diarrheal diseases.

Cryptosporidiosis is an opportunistic infection and individuals with underdeveloped immune systems, such as children under 5 years, and immunocompromised individuals with HIV co-infection are at a higher risk of infection and mortality. Malnutrition in early childhood is also associated with persistent diarrhoea and Cryptosporidium infection, Lancet Infect. Dis., 2015, 15, 85-94.

Nitazoxanide is the only FDA-approved drug for the treatment of Cryptosporidiosis. It has been established that the efficacy of Nitazoxanide is suboptimal and is not an effective treatment for all Cryptosporidium-only infected patients, J. Infect. Dis., 2001, 184, 103-06 and Clin. Gastroenterol. Hepatol., 2006, 4, 320-24. Nitazoxanide was also shown to be ineffective in clinical trials for Cryptosporidium-HIV co-infected patients, who were not co-treated with HIV antiretroviral therapy, Trans. R. Soc. Trop. Med. Hyg. 1998, 92, 663-66 and BMC Infect. Dis. 2009, 9, 195.

Thus there is a need for new anti-infective agents which are effective against Cryptosporidium, and particularly for agents which are suitable for use in the treatment or prevention of; Cryptosporidium-only infected subjects; immunocompromised subjects infected with Cryptosporidium such as Cryptosporidium-HIV co-infected subjects, malnourished children of less than a year suffering from diarrhoea caused by Cryptosporidium infections.

Lysyl t-RNA Synthetase (LysRS or KRS)

Protein synthesis is a complex, multi-step process involving many enzymes. Aminoacyl-tRNA synthetases (aaRS) catalyse the attachment of amino acids to their cognate transfer RNAs playing a key role in protein translation. Inhibition of aaRS has been successfully exploited against bacterial infections with one aaRS inhibitor, mupirocin, currently in clinical use for the topical treatment of methicillin-resistant Staphylococcus aureus (MRSA) infections. Mupirocin is an inhibitor of isoleulcyl-tRNA synthetase (IleRS).

Recently, cladosporin was identified as an inhibitor of lysyl-tRNA syntethase (LysRS). As reported by Scott et. al., J. Antibiot. 1971 24, 747-755 cladosporin is a fungal secondary metabolite. Cladosporin has been shown to inhibit PfLysRS with more than 100-fold selectivity vs HsLysRS, Hoepfner et al, Cell Host Microbe, 2012, 11(6):654-63. We reported that cladosporin and chromone analogues are inhibitors of C. parvum LysRS, Baragana et al., PNAS, 2019, 116 (4), 7015-7020.

A chromone analogue showed reduction of oocyst shedding in two different mouse models of C. parvum infection, WO2017221002 A1 and Baragana et al., PNAS, 2019, 116 (4), 7015-7020. These results validate C. parvum LysRS as a promising mode of action for the treatment of cryptosporidiosis infections.

### SUMMARY

The present invention relates to a compound of Formula I:

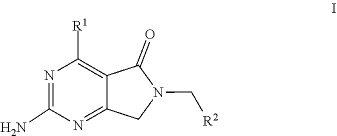

wherein R¹ is selected from: —CH₃, —CF₂H or —CF₃; and
wherein R² is selected from:

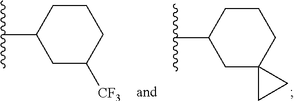 [SMILES: *C1CCCC(C(F)(F)F)C1 ; *C1CCCC2(CC2)C1]

or a veterinarily or pharmaceutically acceptable, salt, hydrate, solvate, isomer, prodrug or polymorph thereof.

In the compound of Formula I, R¹ may be preferably selected from: —CH₃ or —CF₃. Most preferably, R¹ may be —CF₃.

In the compound of Formula I, R² may preferably be

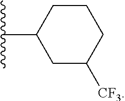 [SMILES: *C1CCCC(C(F)(F)F)C1]

The compound of Formula I may be a compound of Formula I-A:

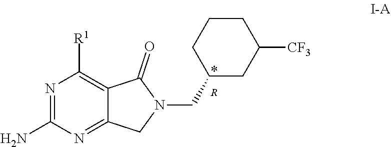

wherein R¹ is selected from: —CH₃, —CF₂H or —CF₃;
or a veterinarily or pharmaceutically acceptable, salt, hydrate, solvate, isomer, prodrug or polymorph thereof.

In the compound of Formula I-A, R¹ may be preferably selected from: —CH₃ or —CF₃. Most preferably, R¹ may be —CF₃.

The compound of Formula I may be a compound of Formula I-B:

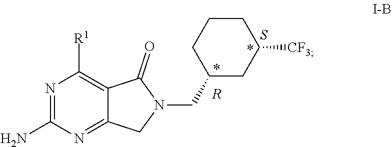

and
wherein R¹ is selected from: —CH₃, —CF₂H or —CF₃;
or a veterinarily or pharmaceutically acceptable, salt, hydrate, solvate, isomer, prodrug or polymorph thereof.

In the compound of Formula I-B, R¹ may be preferably selected from: —CH₃ or —CF₃. Most preferably, R¹ may be —CF₃.

The compound of Formula I may be a compound of Formula I-C:

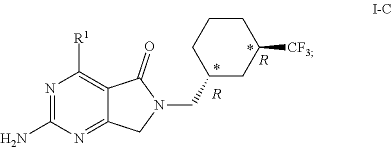

and
wherein R¹ is selected from: —CH₃, —CF₂H or —CF₃;
or a veterinarily or pharmaceutically acceptable, salt, hydrate, solvate, isomer, prodrug or polymorph thereof.

In the compound of Formula I-C, R¹ may be preferably selected from: —CH₃ or —CF₃. Most preferably, R¹ may be —CF₃.

The compound of Formula I may be a compound of Formula I-D:

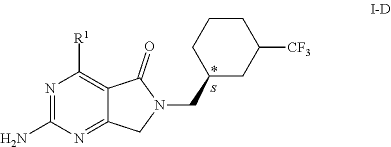

wherein R¹ is selected from: —CH₃, —CF₂H or —CF₃;
or a veterinarily or pharmaceutically acceptable, salt, hydrate, solvate, isomer, prodrug or polymorph thereof.

In the compound of Formula I-D, R¹ may be preferably selected from: —CH₃ or —CF₃. Most preferably, R¹ may be —CF₃.

The compound of Formula I may be a compound of Formula I-E:

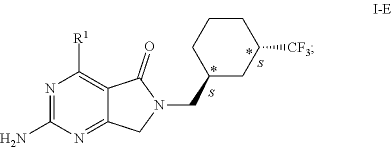

and
wherein R¹ is selected from: —CH₃, —CF₂H or —CF₃;
or a veterinarily or pharmaceutically acceptable, salt, hydrate, solvate, isomer, prodrug or polymorph thereof.

In the compound of Formula I-E, R¹ may be preferably selected from: —CH₃ or —CF₃. Most preferably, R¹ may be —CF₃.

The compound of Formula I may be a compound of Formula I-F:

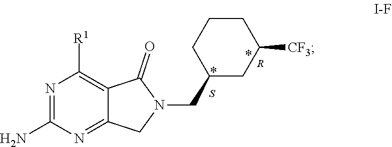

and
wherein R¹ is selected from: —CH₃, —CF₂H or —CF₃;
or a veterinarily or pharmaceutically acceptable, salt, hydrate, solvate, isomer, prodrug or polymorph thereof.

In the compound of Formula I-F, R¹ may be preferably selected from: —CH₃ or —CF₃. Most preferably, R¹ may be —CF₃.

The compound of Formula I may be a compound of Formula l-G:

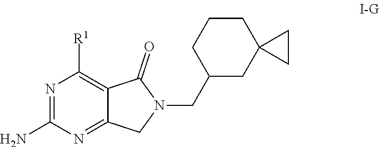

wherein R¹ is selected from: —CH₃, —CF₂H or —CF₃;
or a veterinarily or pharmaceutically acceptable, salt, hydrate, solvate, isomer, prodrug or polymorph thereof.

In some embodiments, the compound of Formula I-G may be a racemic mixture. In some embodiments, the compound of Formula I-G may be optically active.

In the compound of Formula I-G, R¹ may be preferably selected from: —CH₃ or —CF₃. Most preferably, R¹ may be —CF₃.

Preferred compounds of Formula I may be selected from:

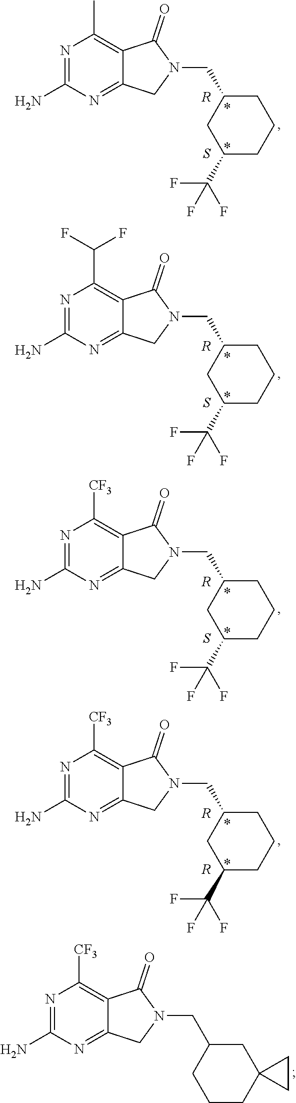

or a veterinarily or pharmaceutically acceptable, salt, hydrate, solvate, isomer, prodrug or polymorph thereof.

Preferred compounds of Formula I may be selected from:
2-amino-4-(trifluoromethyl)-6-(((1R,3S)-3-(trifluoromethyl)cyclohexyl)methyl)-6,7-dihydro-5H-pyrrolo[3,4-d]pyrimidin-5-one;
2-amino-4-methyl-6-(((1R,3S)-3-(trifluoromethyl)cyclohexyl)methyl)-6,7-dihydro-5H-pyrrolo[3,4-d]pyrimidin-5-one;
2-amino-4-(difluoromethyl)-6-(((1R,3S)-3-(trifluoromethyl)cyclohexyl)methyl)-6,7-dihydro-5H-pyrrolo[3,4-d]pyrimidin-5-one;
2-amino-6-(spiro[2.5]octan-5-ylmethyl)-4-(trifluoromethyl)-6,7-dihydro-5H-pyrrolo[3,4-d]pyrimidin-5-one;
2-amino-4-(trifluoromethyl)-6-(((1R,3R)-3-(trifluoromethyl)cyclohexyl)methyl)-6,7-dihydro-5H-pyrrolo[3,4-d]pyrimidin-5-one;
2-amino-4-(trifluoromethyl)-6-(((1 S,3R)-3-(trifluoromethyl)cyclohexyl)methyl)-6,7-dihydro-5H-pyrrolo[3,4-d]pyrimidin-5-one;
2-amino-4-(trifluoromethyl)-6-(((1 S,3S)-3-(trifluoromethyl)cyclohexyl)methyl)-6,7-dihydro-5H-pyrrolo[3,4-d]pyrimidin-5-one;
   or a veterinarily or pharmaceutically acceptable, salt, hydrate, solvate, isomer, prodrug or polymorph thereof.

The compounds of Formula I, I-A, I-B, I-C, I-D, I-E, I-F and/or I-G may have a pEC₅₀ for Cryptosporidium parvum of 6 or more, preferably 6.5 or more, more preferably 7 or more, especially 7.3 or 7.7 or more.

The compounds of Formula I, I-A, I-B, I-C, I-D, I-E, I-F and/or I-G may have a pIC₅₀ for Cryptosporidium parvum lysyl t-RNA synthetase (Cp KRS) of 5 or more, preferably 5.5 or more, more preferably 6 or more.

In a second aspect there is provided a compound of Formula I, I-A, I-B, I-C, I-D, I-E, I-F and/or I-G as described hereinabove, for use in medicine.

In a third aspect there is provided a compound of Formula I, I-A, I-B, I-C, I-D, I-E, I-F and/or I-G as described hereinabove, for veterinary use.

In a fourth aspect there is provided a compound of Formula I, I-A, I-B, I-C, I-D, I-E, I-F and/or I-G as described hereinabove for use as a medicament.

In a further aspect there is provided a compound of 1, I-A, I-B, I-C, I-D, I-E, I-F and/or I-G as described hereinabove for use in the treatment of an infectious disease.

In a further aspect there is provided a compound of Formula I, I-A, I-B, I-C, I-D, I-E, I-F and/or I-G as described hereinabove for use in the treatment of Cryptosporidiosis.

In a further aspect there is provided a compound of Formula I, I-A, I-B, I-C, I-D, I-E, I-F and/or I-G as described hereinabove for use in the treatment of a disease caused by Cryptosporidium. The disease may be caused by a Cryptosporidium species selected from: Cryptosporidium andersoni, Cryptosporidium baileyi, Cryptosporidium bovis, Cryptosporidium canis, Cryptosporidium chipmunk, Cryptosporidium cuniculus, Cryptosporidium ducismarci, Cryptosporidium felis, Cryptosporidium fayeri, Cryptosporidium galli, Cryptosporidium meleagridis, Cryptosporidium muris, Cryptosporidium monari, Cryptosporidium suis, Cryptosporidium scrofarum, Cryptosporidium tyzzeri, Cryptosporidium ubiquitum, Cryptosporidium viatorum, Cryptosporidium nasorum, Cryptosporidium parvum, Cryptosporidium hominis, Cryptosporidium saurophilum, Cryptosporidium serpentis, and Cryptosporidium wrairi.

Another aspect relates to a method for the treatment of a Cryptosporidiosis infection in a human or animal in need thereof. The method comprises administering to the human or animal a therapeutically effective amount of a compound of Formula I, I-A, I-B, I-C, I-D, I-E, I-F and/or I-G as described hereinabove or a pharmaceutically acceptable salt thereof.

Another aspect of this invention relates to a method for the treatment of a disease caused by infection with a Cryptosporidium parasite in a human or animal in need thereof, comprising administering to the human a therapeutically effective amount of a compound of Formula I, I-A, I-B, I-C, I-D, I-E, I-F and/or I-G as described hereinabove or a pharmaceutically acceptable salt thereof.

Another aspect of this invention relates to a use of a compound of Formula I, I-A, I-B, I-C, I-D, I-E, I-F and/or I-G as described hereinabove or a pharmaceutically acceptable salt thereof, in the manufacture of a medicament for use in the treatment of a Cryptosporidiosis infection or a disease caused by infection with a Cryptosporidium parasite.

Another aspect of this invention relates to a combination of (a) a compound of Formula I, I-A, I-B, I-C, I-D, I-E, I-F and/or I-G as described hereinabove or a pharmaceutically acceptable salt thereof; and (b) at least one other anti-parasitic agent.

Another aspect of this invention relates to a kit comprising a compound of Formula I, I-A, I-B, I-C, I-D, I-E, I-F and/or I-G as described hereinabove or a pharmaceutically acceptable salt thereof and instructions for administering to a human or animal in need thereof.

### DETAILED DESCRIPTION

### Terms and Definitions

It will be understood that a phrase such as “a compound of Formula I, I-A, I-B, I-C, I-D, I-E, I-F and/or I-G or a pharmaceutically acceptable salt thereof” is intended to encompass the compound of Formula I, I-A, I-B, I-C, I-D, I-E, I-F and/or I-G, a pharmaceutically acceptable salt or solvate of the compound of Formula I, I-A, I-B, I-C, I-D, I-E, I-F and/or I-G, or any pharmaceutically acceptable combination of these.

Thus by way of non-limiting example used here for illustrative purpose, “a compound of Formula I, I-A, I-B, I-C, I-D, I-E, I-F and/or I-G or a pharmaceutically acceptable salt thereof” encompasses a pharmaceutically acceptable salt of a compound of Formula I, I-A, I-B, I-C, I-D, I-E, I-F and/or I-G which is present as a solvate or a hydrate, and this phrase also encompasses a mixture of a compound of Formula I, I-A, I-B, I-C, I-D, I-E, I-F and/or I-G and a pharmaceutically acceptable salt of a compound of Formula I, I-A, I-B, I-C, I-D, I-E, I-F and/or I-G.

It is to be further understood that references herein refer to a compound of Formula I, I-A, I-B, I-C, I-D, I-E, I-F and/or I-G or a pharmaceutically acceptable salt thereof includes a compound of Formula I, I-A, I-B, I-C, I-D, I-E, I-F and/or I-G as a free base or as a pharmaceutically acceptable salt thereof.

The term “pharmaceutically acceptable” refers to those compounds (including salts), materials, compositions, and dosage forms which are, within the scope of sound medical judgment, suitable for use in contact with the tissues of human beings and animals without excessive toxicity, irritation, or other problem or complication, commensurate with a reasonable benefit/risk ratio.

As used herein, the term “pharmaceutically acceptable salts” represents salts that retain the desired biological activity of the human compound and exhibit minimal undesired toxicological effects. These pharmaceutically acceptable salts may be prepared in situ during the final isolation and purification of the compound, or by separately reacting the purified compound in its free acid or free base form with a suitable base or acid, respectively.

Pharmaceutically acceptable salts include, amongst others, those described in Berge, J. Pharm. Sci., 1977, 66, 1-19, or those listed in P H Stahl and C G Wermuth, editors, Handbook of Pharmaceutical Salts; Properties, Selection and Use, Second Edition Stahl/Wermuth: Wiley-VCH/VHCA, 2011 (see http://www.wiley.com/WileyCDA/WileyTitle/productCd-3906390519.html).

Where the compound functionality allows, suitable pharmaceutically acceptable salts of the compound of Formula I, I-A, I-B, I-C, I-D, and/or I-E can be formed, which include acid addition salts. Acid addition salts may be formed by reaction with the appropriate acid, optionally in a suitable solvent such as an organic solvent, to give the salt which can be isolated by crystallisation and filtration. Representative pharmaceutically acceptable acid addition salts include, but are not limited to, 4-acetamidobenzoate, acetate, adipate, alginate, ascorbate, aspartate, benzenesulfonate (besylate), benzoate, bisulfate, bitartrate, butyrate, calcium edetate, camphorate, camphorsulfonate (camsylate), caprate (decanoate), caproate (hexanoate), caprylate (octanoate), cinnamate, citrate, cyclamate, digluconate, 2,5-dihydroxybenzoate, disuccinate, dodecylsulfate (estolate), edetate (ethylenediaminetetraacetate), estolate (lauryl sulfate), ethane-1,2-disulfonate (edisylate), ethanesulfonate (esylate), formate, fumarate, galactarate (mucate), gentisate (2,5-dihydroxybenzoate), glucoheptonate (gluceptate), gluconate, glucuronate, glutamate, glutarate, glycerophosphorate, glycolate, hexylresorcinate, hippurate, hydrabamine (N,N′-di(dehydroabietyl)-ethylenediamine), hydrobromide, hydrochloride, hydroiodide, hydroxynaphthoate, isobutyrate, lactate, lactobionate, laurate, malate, maleate, malonate, mandelate, methanesulfonate (mesylate), methylsulfate, mucate, naphthalene-1,5-disulfonate (napadisylate), naphthalene-2-sulfonate (napsylate), nicotinate, nitrate, oleate, palmitate, p-aminobenzenesulfonate, p-aminosalicyclate, pamoate (embonate), pantothenate, pectinate, persulfate, phenylacetate, phenylethylbarbiturate, phosphate, polygalacturonate, propionate, p-toluenesulfonate (tosylate), pyroglutamate, pyruvate, salicylate, sebacate, stearate, subacetate, succinate, sulfamate, sulfate, tannate, tartrate, teoclate (8-chlorotheophyllinate), thiocyanate, triethiodide, undecanoate, undecylenate, and valerate.

As used herein, the term “therapeutically effective amount” means any amount which, as compared to a corresponding human who has not received such amount, results in improved treatment, healing, prevention, or amelioration of a disease, disorder, or side effect, or a decrease in the rate of advancement of a disease or disorder. An appropriate “therapeutically effective amount” will depend upon a number offactors including, for example, the age and weight of the human, the precise condition requiring treatment and its severity, the nature of the formulation, and the route of administration, and will ultimately be at the discretion of the attendant physician.

As used herein, “treatment” and “treating”, are used interchangeably herein, and refer to an approach for obtaining beneficial or desired results including, but not limited to, therapeutic benefit. By therapeutic benefit in meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient can still be afflicted with the underlying disorder. The term “treat’, in all its verb forms, is used herein to mean to relieve, alleviate, prevent, and/or manage at least one symptom of a disorder in a human.

As used herein, the term “solvate” refers to forms of the compound of 1, I-A, I-B, I-C, I-D, I-E, I-F and/or I-G, or a pharmaceutically acceptable salt thereof, that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates, ethanolates, and methanolates. In some embodiments, the compounds of Formula I, I-A, I-B, I-C, I-D, I-E, I-F and/or I-G or pharmaceutically acceptable salts thereof may be isolated, formed, and/or administered as a solvate.

As used herein, the term “hydrate” refers to forms of the compound of Formula I, I-A, I-B, I-C, I-D, I-E, I-F and/or I-G, or a pharmaceutically acceptable salt thereof, that are associated with water. Typically, the number of the water molecules contained in a hydrate of a compound is in a definite ratio to the number of the compound molecules in the hydrate. Therefore, a hydrate of a compound may be represented, for example, by the general formula R·x H2O, wherein R is the compound, and x is a number greater than 0. A given compound may form more than one type of hydrate, including, e.g., monohydrates (x is 1), lower hydrates (x is a number greater than 0 and smaller than 1, e.g., hemihydrates (R·0.5 H2O)), and polyhydrates (x is a number greater than 1, e.g., dihydrates (R·2 H2O) and hexahydrates (R·6 H2O)). In some embodiments, the compounds of Formula I, I-A, I-B, I-C, I-D, I-E, I-F and/or I-G or pharmaceutically acceptable salts thereof may be isolated, formed, and/or administered as a hydrate.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric centre, for example, it is bonded to four different groups, a pair of enantiomers is possible. An enantiomer can be characterized by the absolute configuration of its asymmetric centre and is described by the R- and S-sequencing rules of Cahn and Prelog, or by the manner in which the molecule rotates the plane of polarized light and designated as dextrorotatory or levorotatory {i.e., as (+) or (−)-isomers respectively). A chiral compound can exist as either individual enantiomer or as a mixture thereof. A mixture containing equal proportions of the enantiomers is called a “racemic mixture”. In some embodiments, the compounds of Formula (I) or pharmaceutically acceptable salts thereof, can contain one or more asymmetric centres (also referred to as a chiral centres) and may, therefore, exist as individual enantiomers, diastereoisomers, or other stereoisomeric forms, or as mixtures thereof. Reference herein to compounds of Formula I or pharmaceutically acceptable salts thereof refer to any single or mixture of stereoisomers unless otherwise designated.

The term “polymorph” refers to a crystalline form of a compound (or a salt, hydrate, or solvate thereof). All polymorphs have the same elemental composition. Different crystalline forms usually have different X-ray diffraction patterns, infrared spectra, melting points, density, hardness, crystal shape, optical and electrical properties, stability, and solubility. Recrystallization solvent, rate of crystallization, storage temperature, and other factors may cause one crystal form to dominate. Various polymorphs of a compound can be prepared by crystallization under different conditions. Polymorphic forms of a compound of Formula (I) or a pharmaceutically acceptable salt thereof may be characterised and differentiated using a number of conventional analytical techniques, including, but not limited to, X-ray powder diffraction (XRPD), infrared spectroscopy (IR), Raman spectroscopy, differential scanning calorimetry (DSC), thermogravimetric analysis (TGA) and solid-state nuclear magnetic resonance (ssNMR).

### Compounds

This invention relates to a compound of Formula I:

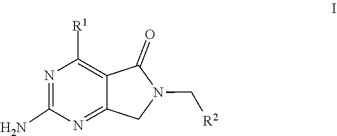

wherein R¹ is selected from: —CH₃, —CF₂H or —CF₃; and
wherein R² is selected from:

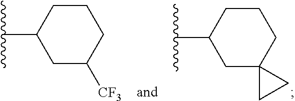 [SMILES: *C1CCCC(C(F)(F)F)C1 ; *C1CCCC2(CC2)C1]

or a veterinarily or pharmaceutically acceptable, salt, hydrate, solvate, isomer, prodrug or polymorph thereof.

In the compound of Formula I, R¹ may be preferably selected from: —CH₃ or —CF₃. Most preferably, R¹ may be —CF₃.

In the compound of Formula I, R² may preferably be

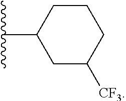 [SMILES: *C1CCCC(C(F)(F)F)C1]

The compound of Formula I may be a compound of Formula I-A:

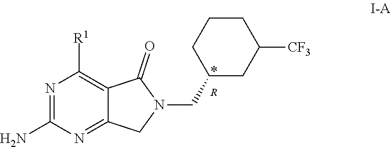

wherein R¹ is selected from: —CH₃, —CF₂H or —CF₃;
or a veterinarily or pharmaceutically acceptable, salt, hydrate, solvate, isomer, prodrug or polymorph thereof.

In the compound of Formula I-A, R¹ may be preferably selected from: —CH₃ or —CF₃. Most preferably, R¹ may be —CF₃.

The compound of Formula I may be a compound of Formula I-B:

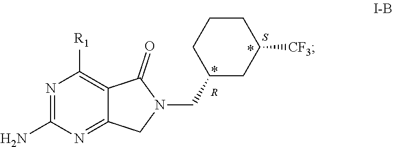

and
wherein R¹ is selected from: —CH₃, —CF₂H or —CF₃;
or a veterinarily or pharmaceutically acceptable, salt, hydrate, solvate, isomer, prodrug or polymorph thereof.

In the compound of Formula I-B, R¹ may be preferably selected from: —CH₃ or —CF₃. Most preferably, R¹ may be —CF₃.

The compound of Formula I may be a compound of Formula I-C:

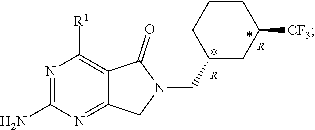

and
wherein R¹ is selected from: —CH₃, —CF₂H or —CF₃;
or a veterinarily or pharmaceutically acceptable, salt, hydrate, solvate, isomer, prodrug or polymorph thereof.

In the compound of Formula I-C, R¹ may be preferably selected from: —CH₃ or —CF₃. Most preferably, R¹ may be —CF₃.

The compound of Formula I may be a compound of Formula I-D:

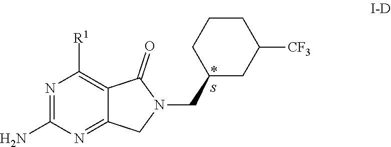

wherein R¹ is selected from: —CH₃, —CF₂H or —CF₃;
or a veterinarily or pharmaceutically acceptable, salt, hydrate, solvate, isomer, prodrug or polymorph thereof.

In the compound of Formula I-D, R¹ may be preferably selected from: —CH₃ or —CF₃. Most preferably, R¹ may be —CF₃.

The compound of Formula I may be a compound of Formula I-E:

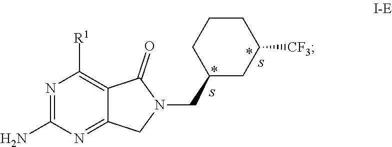

and
wherein R¹ is selected from: —CH₃, —CF₂H or —CF₃;
or a veterinarily or pharmaceutically acceptable, salt, hydrate, solvate, isomer, prodrug or polymorph thereof.

In the compound of Formula I-E, R¹ may be preferably selected from: —CH₃ or —CF₃. Most preferably, R¹ may be —CF₃.

The compound of Formula I may be a compound of Formula I-F:

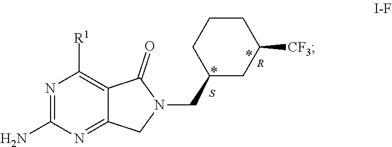

and
wherein R¹ is selected from: —CH₃, —CF₂H or —CF₃;
or a veterinarily or pharmaceutically acceptable, salt, hydrate, solvate, isomer, prodrug or polymorph thereof.

In the compound of Formula I-F, R¹ may be preferably selected from: —CH₃ or —CF₃. Most preferably, R¹ may be —CF₃.

The compound of Formula I may be a compound of Formula I-G:

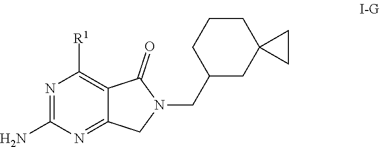

wherein R¹ is selected from: —CH₃, —CF₂H or —CF₃;
or a veterinarily or pharmaceutically acceptable, salt, hydrate, solvate, isomer, prodrug or polymorph thereof.

In the compound of Formula I-G, R¹ may be preferably selected from: —CH₃ or —CF₃. Most preferably, R¹ may be —CF₃.

Exemplary compounds of Formula I are depicted in Table 1.

TABLE 1
Exemplary compounds of Formula 1

| Compound | | |
|---|---|---|
| No. | Structure | Chemical Name |
| 1 | 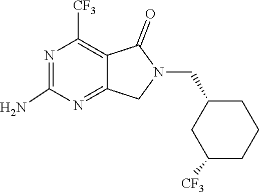 [SMILES: Nc1nc2c(c(C(F)(F)F)n1)C(=O)N(C[C@@H]1CCC[C@H](C(F)(F)F)C1)C2] | 2-amino-4-(trifluoromethyl)-6-(((1R,3S)-3- (trifluoromethyl)cyclohexyl)methyl)-6,7- dihydro-5H-pyrrolo[3,4-d]pyrimidin-5-one |
| 2 | 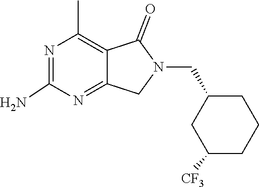 [SMILES: Cc1nc(N)nc2c1C(=O)N(C[C@@H]1CCC[C@H](C(F)(F)F)C1)C2] | 2-amino-4-methyl-6-(((1R,3S)-3- (trifluoromethyl)cyclohexyl)methyl)-6,7- dihydro-5H-pyrrolo[3,4-d]pyrimidin-5-one |
| 3 | 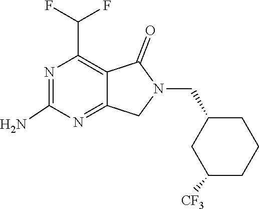 [SMILES: Nc1nc2c(c(C(F)F)n1)C(=O)N(C[C@@H]1CCC[C@H](C(F)(F)F)C1)C2] | 2-amino-4-(difluoromethyl)-6-(((1R,3S)-3- (trifluoromethyl)cyclohexyl)methyl)-6,7- dihydro-5H-pyrrolo[3,4-d]pyrimidin-5-one |
| 4 | 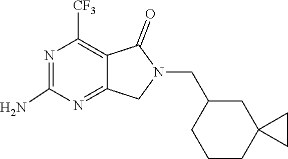 [SMILES: Nc1nc2c(c(C(F)(F)F)n1)C(=O)N(CC1CCCC3(CC3)C1)C2] | Racemic 2-amino-6-(spiro[2.5]octan-5- ylmethyl)-4-(trifluoromethyl)-6,7-dihydro- 5H-pyrrolo[3,4-d]pyrimidin-5-one |
| 5 | 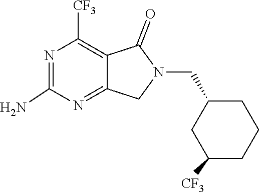 [SMILES: Nc1nc2c(c(C(F)(F)F)n1)C(=O)N(C[C@@H]1CCC[C@@H](C(F)(F)F)C1)C2] | 2-amino-4-(trifluoromethyl)-6-(((1R,3R)-3- (trifluoromethyl)cyclohexyl)methyl)-6,7- dihydro-5H-pyrrolo[3,4-d]pyrimidin-5-one |
| 6 | 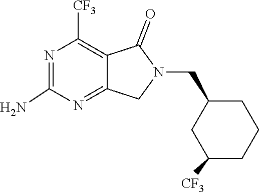 [SMILES: Nc1nc2c(c(C(F)(F)F)n1)C(=O)N(C[C@H]1CCC[C@@H](C(F)(F)F)C1)C2] | 2-amino-4-(trifluoromethyl)-6-(((1S,3R)-3- (trifluoromethyl)cyclohexyl)methyl)-6,7- dihydro-5H-pyrrolo[3,4-d]pyrimidin-5-one |
| 7 | 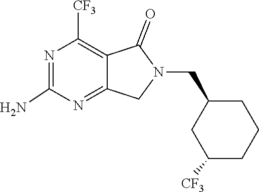 [SMILES: Nc1nc2c(c(C(F)(F)F)n1)C(=O)N(C[C@H]1CCC[C@H](C(F)(F)F)C1)C2] | 2-amino-4-(trifluoromethyl)-6-(((1S,3S)-3- (trifluoromethyl)cyclohexyl)methyl)-6,7- dihydro-5H-pyrrolo[3,4-d]pyrimidin-5-one |

Preferred compounds of Formula I may be selected from:

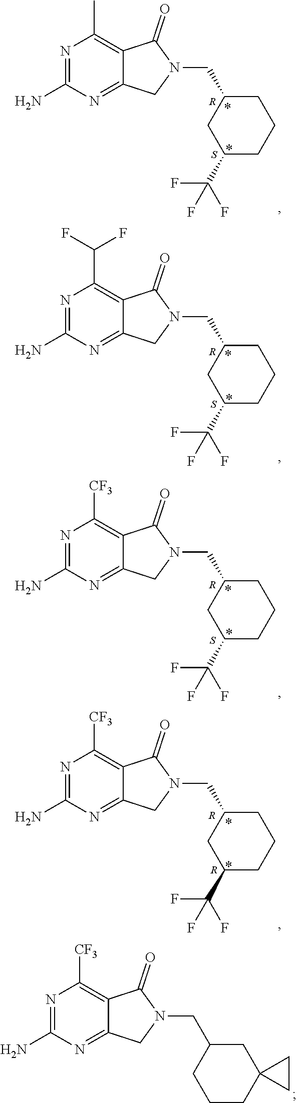

or a veterinarily or pharmaceutically acceptable, salt, hydrate, solvate, isomer, prodrug or polymorph thereof.

Preferred compounds of Formula I may be selected from:
2-amino-4-(trifluoromethyl)-6-(((1R,3S)-3-(trifluoromethyl)cyclohexyl)methyl)-6,7-dihydro-5H-pyrrolo[3,4-d]pyrimidin-5-one;
2-amino-4-methyl-6-(((1R,3S)-3-(trifluoromethyl)cyclohexyl)methyl)-6,7-dihydro-5H-pyrrolo[3,4-d]pyrimidin-5-one;
2-amino-4-(difluoromethyl)-6-(((1R,3S)-3-(trifluoromethyl)cyclohexyl)methyl)-6,7-dihydro-5H-pyrrolo[3,4-d]pyrimidin-5-one; and
2-amino-6-(spiro[2.5]octan-5-ylmethyl)-4-(trifluoromethyl)-6,7-dihydro-5H-pyrrolo[3,4-d]pyrimidin-5-one;
2-amino-4-(trifluoromethyl)-6-(((1R,3R)-3-(trifluoromethyl)cyclohexyl)methyl)-6,7-dihydro-5H-pyrrolo[3,4-d]pyrimidin-5-one;
   or a veterinarily or pharmaceutically acceptable, salt, hydrate, solvate, isomer, prodrug or polymorph thereof.

Particularly preferred compounds of Formula I may be selected from:
2-amino-4-(trifluoromethyl)-6-(((1R,3S)-3-(trifluoromethyl)cyclohexyl)methyl)-6,7-dihydro-5H-pyrrolo[3,4-d]pyrimidin-5-one;
2-amino-4-(difluoromethyl)-6-(((1R,3S)-3-(trifluoromethyl)cyclohexyl)methyl)-6,7-dihydro-5H-pyrrolo[3,4-d]pyrimidin-5-one; and
2-amino-4-methyl-6-(((1R,3S)-3-(trifluoromethyl)cyclohexyl)methyl)-6,7-dihydro-5H-pyrrolo[3,4-d]pyrimidin-5-one;
   or a veterinarily or pharmaceutically acceptable, salt, hydrate, solvate, isomer, prodrug or polymorph thereof.

The compounds of Formula I, I-A, I-B, I-C, I-D, I-E, I-F and/or I-G may have a pEC₅₀ for Cryptosporidium parvum of 6 or more, preferably 6.5 or more, more preferably 7 or more, especially 7.3 or 7.7 or more.

The compounds of Formula I, I-A, I-B, I-C, I-D, I-E, I-F and/or I-G may have a pIC₅₀ for Cryptosporidium parvum lysyl t-RNA synthetase (Cp KRS) of 5 or more, preferably 5.5 or more, more preferably 6 or more.

It will be appreciated that a compound of Formula I, I-A, I-B, I-C, I-D, I-E, I-F and/or I-G may exist in different tautomeric forms. All possible tautomers, and pharmaceutically acceptable salts thereof, are contemplated to be within the scope of the present invention.

The compound of Formula I, I-A, I-B, I-C, I-D, I-E, I-F and/or I-G or pharmaceutically acceptable salt thereof may be in crystalline or amorphous forms. Furthermore, some of the crystalline forms may exist as polymorphs, all of which are included within the scope of the present invention. The most thermodynamically stable polymorphic form or forms of a compound of Formula I, I-A, I-B, I-C, I-D, and/or I-E or a pharmaceutically acceptable salt thereof are of particular interest. In one aspect of the invention, a compound of Formula I, I-A, I-B, I-C, I-D, I-E, I-F and/or I-G or pharmaceutically acceptable salt thereof is crystalline.

### Methods of Use

It is to be appreciated that references to treatment as used herein includes prophylaxis as well as palliative treatment via the alleviation of established symptoms of a condition i.e. prevention or control. “Treating” or “treatment” of a state, disorder or condition includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i. e., causing regression of the state, disorder or condition or at least one of its clinical or subclinical symptoms.

It will be appreciated by those skilled in the art that references herein to treatment refer to the treatment of established conditions. However, the compounds of general formula (I) and pharmaceutically acceptable salts thereof may, depending on the condition, also be useful in the prevention (prophylaxis) of certain diseases.

As used herein, unless otherwise indicated, “treat”, “treating” or “treatment” in reference to a disease means: (1) to ameliorate the disease or one or more of the biological manifestations of the disease (2) to interfere with (a) one or more points in the biological cascade that leads to or is responsible for the disease or (b) one or more of the biological manifestations of the disease, (3) to alleviate one or more of the symptoms or effects associated with the disease, (4) to slow the progression of the disease or one or more of the biological manifestations of the disease, and/or (5) to diminish the likelihood of severity of a disease or biological manifestations of the disease.

As used herein, unless otherwise indicated, “prevent”, “preventing” or “prevention” means the prophylactic administration of a drug to diminish the likelihood of the onset of or to delay the onset of a disease or biological manifestation thereof. The skilled artisan will appreciate that “prevention” is not an absolute term. In medicine, “prevention” is understood to refer to the prophylactic administration of a drug to substantially diminish the likelihood or severity of a disorder or biological manifestation thereof, or to delay the onset of such disorder or biological manifestation thereof.

Thus, in one embodiment, there is provided the treatment or prevention of a disease. In another embodiment, there is provided the treatment of a disease. In a further embodiment, there is provided the prevention of a disease.

There is thus provided as a further aspect of the invention a compound of Formula I, or a pharmaceutically acceptable salt thereof, for use in therapy. There is further provided a compound of general formula (I), or a pharmaceutically acceptable salt thereof, for use as a medicament in therapy, which therapy is human or veterinary.

It will be appreciated that, when a compound of Formula I or a pharmaceutically acceptable salt thereof is used in therapy, it is used as an active therapeutic agent.

For the avoidance of doubt, general references herein to “treatment” include references to curative, palliative and prophylactic treatment.

### Human and Veterinary Use

Regarding the use of the compounds of the invention in humans, there is provided:
a pharmaceutical composition comprising a compound of Formula I, or a pharmaceutically acceptable, salt, solvate, hydrate, isomer, prodrug or polymorph thereof, together with one or more pharmaceutically acceptable, carrier, diluent or excipient;
a compound of Formula I, or a pharmaceutically acceptable, salt, solvate, hydrate, isomer, prodrug or polymorph thereof, or a pharmaceutical composition containing any of the foregoing, for use as a medicament;
a compound of Formula I, or a pharmaceutically acceptable, salt, solvate, hydrate, isomer, prodrug or polymorph thereof, or a pharmaceutical composition containing any of the foregoing, for use in medicine;
a compound of Formula I, or a pharmaceutically acceptable, salt, solvate, hydrate, isomer, prodrug or polymorph thereof, or a pharmaceutical composition containing any of the foregoing, for use in the treatment of an infectious disease. Preferably, the infectious disease may be an infectious disease caused by Cryptosporidium. The infectious disease may be caused by a Cryptosporidium species selected from: Cryptosporidium andersoni, Cryptosporidium baileyi, Cryptosporidium bovis, Cryptosporidium canis, Cryptosporidium chipmunk, Cryptosporidium cuniculus, Cryptosporidium ducismarci, Cryptosporidium felis, Cryptosporidium fayeri, Cryptosporidium galli, Cryptosporidium meleagridis, Cryptosporidium muris, Cryptosporidium monari, Cryptosporidium suis, Cryptosporidium scrofarum, Cryptosporidium tyzzeri, Cryptosporidium ubiquitum, Cryptosporidium viatorum, Cryptosporidium nasorum, Cryptosporidium parvum, Cryptosporidium hominis, Cryptosporidium saurophilum, Cryptosporidium serpentis, and Cryptosporidium wrairi.a compound of Formula I, or a pharmaceutically acceptable, salt, solvate, hydrate, isomer, prodrug or polymorph thereof, or a pharmaceutical composition containing any of the foregoing, for use in the prophylactic treatment of an infectious disease. Preferably, the infectious disease may be an infectious disease caused by Cryptosporidium. The infectious disease may be caused by a Cryptosporidium species selected from: Cryptosporidium andersoni, Cryptosporidium baileyi, Cryptosporidium bovis, Cryptosporidium canis, Cryptosporidium chipmunk, Cryptosporidium cuniculus, Cryptosporidium ducismarci, Cryptosporidium felis, Cryptosporidium fayeri, Cryptosporidium galli, Cryptosporidium meleagridis, Cryptosporidium muris, Cryptosporidium monari, Cryptosporidium suis, Cryptosporidium scrofarum, Cryptosporidium tyzzeri, Cryptosporidium ubiquitum, Cryptosporidium viatorum, Cryptosporidium nasorum, Cryptosporidium parvum, Cryptosporidium hominis, Cryptosporidium saurophilum, Cryptosporidium serpentis, and Cryptosporidium wrairi.Regarding the use of the compounds of the invention in animals, there is provided:
a veterinary composition comprising a compound of Formula I, or an acceptable salt, solvate, hydrate, isomer, prodrug or polymorph thereof, together with one or more acceptable carrier, diluent or excipient;
a compound of Formula I, or an acceptable salt, solvate, hydrate, isomer, prodrug or polymorph thereof, or a veterinary composition containing any of the foregoing, for use as a veterinary medicine.

For the avoidance of doubt, where use of the compounds of Formula I is referred to herein this additional means use of the compounds of any one of the formulae independently selected from: I, I-A, I-B, I-C, I-D, I-E, I-F and/or I-G

### Treatment of Cryptosporidiosis

There is also therefore provided a compound of Formula I, I-A, I-B, I-C, I-D, I-E, I-F and/or I-G, or a pharmaceutically acceptable salt thereof, for use in the treatment or prevention of Cryptosporidiosis.

There is further provided a method of treatment or prevention of Cryptosporidiosis, which method comprises administering to a human subject in need thereof, a therapeutically effective amount of a compound of Formula I, I-A, I-B, I-C, I-D, I-E, I-F and/or I-G, or a veterinarily or pharmaceutically acceptable salt thereof. In another embodiment of the invention there is provided a method of treatment or prevention of Cryptosporidiosis, which method comprises administering to a mammal in need thereof, a therapeutically effective amount of a compound of Formula I, I-A, I-B, I-C, I-D, I-E, I-F and/or I-G, or a veterinarily or pharmaceutically acceptable salt thereof.

According to a preferred aspect the above uses, and/or methods provide compounds of Formula I, I-A, I-B, I-C, I-D, I-E, I-F and/or I-G, or a pharmaceutically acceptable salt thereof effective against Cryptosporidium, and particularly compounds of Formula I, I-A, I-B, I-C, I-D, I-E, I-F and/or I-G, suitable for use in the treatment or prevention of; Cryptosporidium-only infected subjects; immunocompromised subjects infected with Cryptosporidium such as Cryptosporidium-HIV co-infected subjects.

### Pharmaceutical Compositions

According to a further aspect the present invention provides a pharmaceutical composition comprising: a compound of Formula I, I-A, I-B, I-C, I-D, I-E, I-F and/or I-G or a pharmaceutically acceptable, salt, solvate, hydrate, isomer, prodrug, or polymorph thereof; The pharmaceutical composition may further comprise one or more pharmaceutically acceptable, carriers, diluents or excipients. The pharmaceutical composition may further comprise one or more further active agents against Cryptosporidium.

### Administration and Dose Ranges

In order to select the most appropriate dosage forms and routes of administration considered appropriate for the treatment of the desired indication, compounds of Formula I, I-A, I-B, I-C, I-D, I-E, I-F and/or I-G should be assessed for their biopharmaceutical properties, such as for example, solubility, solution stability (across a range of pHs), likely dose level and permeability. Initial biopharmaceutical testing for potential as anti-malarial treatment has provided positive results.

Compounds of the invention intended for pharmaceutical and/or veterinary use may be administered as crystalline or amorphous products. They may be obtained, for example, as solid plugs, powders, or films by methods such as precipitation, crystallization, freeze-drying, spray drying, or evaporative drying. Microwave or radio frequency drying may be used for this purpose.

They may be administered alone or in combination with one or more other compounds of the invention or in combination with one or more other drugs (or as any combination thereof). Generally, they will be administered as a formulation in association with one or more pharmaceutically acceptable excipients. The term ‘excipient’ is used herein to describe any ingredient other than the compound(s) of the invention. The choice of excipient will to a large extent depend on factors such as the particular mode of administration, the effect of the excipient on solubility and stability, and the nature of the dosage form. Pharmaceutically and veterinarily acceptable excipients include one or more of: lubricants, binding agents, diluents, surface-active agents, anti-oxidants, colorants, flavouring agents, preservatives, flavour enhancers, preservatives, salivary stimulating agents, cooling agents, co-solvents (including oils), emollients, bulking agents, anti-foaming agents, surfactants and taste-masking agents.

Pharmaceutical and veterinary compositions suitable for the delivery of compounds of the present invention and methods for their preparation will be readily apparent to those skilled in the art. Such compositions and methods for their preparation may be found, for example, in Remington's Pharmaceutical Sciences, 19th Edition (Mack Publishing Company, 1995).

Formulations suitable for oral administration include solids, semi-solids or liquids such as tablets; soft or hard capsules; bolus; powders; lozenges (including liquid-filled); chews; multi and nano-particulates; gels; solid solutions; fast-dispersing dosage forms; fast-dissolving dosage forms; fast-disintegrating dosage forms; films; ovules; sprays; buccal/mucoadhesive patches; and liquid formulations. Liquid formulations include suspensions, solutions, elixirs and syrups. Oral administration may involve swallowing, so that the compound enters the gastrointestinal tract, and/or buccal, lingual or sublingual administration by which the compound enters the blood stream directly from the mouth. Liquid formulations may be employed as fillers in soft or hard capsules and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

Formulations for oral administration may be formulated to be immediate and/or modified release. Modified release formulations include delayed-, sustained-, pulsed-, controlled-, targeted and programmed release. The formulation of tablets is discussed in “Pharmaceutical Dosage Forms: Tablets, Vol. 1”, by H. Lieberman and L. Lachman, Marcel Dekker, N. Y., N.Y., 1980 (ISBN 0-8247-6918-X).

The present invention provides a pharmaceutical or veterinary composition formulated for oral delivery comprising a compound of Formula I, I-A, I-B, I-C, I-D, I-E, I-F and/or I-G or a pharmaceutically acceptable, salt, solvate or hydrate thereof, according to any preceding claim, together with one or more pharmaceutically acceptable excipients. The present invention further provides said pharmaceutical or veterinary composition formulated for oral delivery as an immediate release, or as a modified release tablet formulation.

The compounds of the invention may also be administered parenterally, or by injection directly into the blood stream, into muscle, or into an internal organ. Suitable means for parenteral administration include intravenous, intraarterial, intraperitoneal, intrathecal, intraventricular, intraurethral, intrasternal, intracranial, intramuscular, intrasynovial and subcutaneous. Suitable devices for parenteral administration include needle (including microneedle) injectors, needle-free injectors and infusion techniques.

The present invention provides a pharmaceutical or veterinary composition formulated for parenteral delivery comprising a compound of Formula I, I-A, I-B, I-C, I-D, I-E, I-F and/or I-G or a pharmaceutically acceptable, salt, solvate or hydrate thereof, according to any preceding claim, together with one or more pharmaceutically acceptable excipients. The present invention further provides said pharmaceutical or veterinary composition formulated for parenteral delivery as an immediate release, or as a modified release tablet formulation suitable for intramuscular or intravenous administration.

The compounds of the invention may also be administered topically, (intra)dermally, or transdermally to the skin or mucosa. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used.

The compounds of the invention may be administered rectally or vaginally, for example, in the form of a suppository, pessary, or enema. Cocoa butter is a traditional suppository base, but various alternatives may be used as appropriate.

Pharmaceutical formulations containing compounds of the invention may be formulated to be immediate and/or modified release. Modified release formulations include delayed-, sustained-, pulsed-, controlled-, targeted and programmed release.

### Dosages

Typically, a physician and/or veterinary will determine the actual dosage which will be most suitable for an individual subject. The specific dose level and frequency of dosage for any particular individual may be varied and will depend upon a variety of factors including the condition being treated, the activity of the specific compound employed, the metabolic stability and length of action of that compound, the species, the age, body weight, general health, sex, diet, mode and time of administration, rate of excretion, drug combination, the severity of the particular condition, and the individual undergoing therapy.

In general however a suitable dose will be in the range of from about 0.001 to about 50 mg/kg of body weight per day, in a further embodiment, of from about 0.001 to about 5 mg/kg of body weight per day; in a further embodiment of from about 0.001 to about 0.5 mg/kg of body weight per day and in yet a further embodiment of from about 0.001 to about 0.1 mg/kg of body weight per day. In further embodiments, the ranges can be of from about 0.001 to about 750 mg/kg of body weight per day, in the range of 0.5 to 60 mg/kg/day, and in the range of 1 to 20 mg/kg/day.

The desired dose may conveniently be presented in a single dose or as divided doses administered at appropriate intervals, for example as one, two, three, four or more doses per day. If the compounds are administered transdermally or in extended release form the compounds could be dosed once a day or less.

The compound is conveniently administered in unit dosage form; for example containing 0.1 to 50 mg, conveniently 0.1 to 10 mg, most conveniently 0.1 to 5 mg of active ingredient per unit dosage form. In yet a further embodiment the compound can be conveniently administered in unit dosage form; for example containing 10 to 1500 mg, 20 to 1000 mg, or 50 to 700 mg of active ingredient per unit dosage form.

These dosages are based on an average human subject having a weight of about 65 kg to 70 kg. The physician will readily be able to determine doses for subjects whose weight falls outside this range, such as infants and the elderly.

The present invention provides a pharmaceutical composition formulated as a single-dose tablet suitable for oral delivery comprising a compound of formula (I) or a pharmaceutically acceptable, salt, solvate or hydrate thereof, together with one or more pharmaceutically acceptable excipients. The present invention further provides said pharmaceutical composition formulated for oral delivery as an immediate release, or as a modified release single-dose tablet formulation.

The present invention further provides a pharmaceutical composition formulated as a single-dose tablet formulated for oral delivery as an immediate release, or as a modified release single-dose tablet formulation comprising from about 0.1 to about 3000 mg, preferably from about 0.5 to about 1500 mg, more preferably from about 1 to about 750 mg, from about 1 to about 750 mg, and especially from about 5 to about 250 mg of a compound of formula (I) or a pharmaceutically acceptable, salt, solvate or hydrate thereof, together with one or more pharmaceutically acceptable excipients.

For anti-infective treatment, of any of the infectious conditions identified hereinbefore, the present invention further provides a pharmaceutical composition formulated as a single-dose tablet formulated for oral delivery as an immediate release, or as a modified release single-dose tablet formulation comprising from 0.1 to 3000 mg, preferably from about 0.5 to about 1500 mg, more preferably from about 1 to about 750 mg and especially from about 5 to about 250 mg of a compound of formula (I) or a pharmaceutically acceptable, salt, solvate or hydrate thereof, together with one or more pharmaceutically acceptable excipients.

Where single treatment therapy via a large dose is to be administered, for example to a child, the dose could be provided by more than one tablet, such as 2×1500 mg, or 3×1000 mg, rather than a single-dose 3000 mg tablet where the tablets may be taken either one after the other, or together according to suitability.

### Co-Administration

Inasmuch as it may desirable to administer a combination of active compounds, as detailed hereinbefore, for example, for the purpose of treating a particular infectious disease, it is within the scope of the present invention that two or more pharmaceutical compositions, at least one of which contains a compound of Formula I, I-A, I-B, I-C, I-D, I-E, I-F and/or I-G as defined hereinbefore in accordance with the invention, may conveniently be combined in the form of a kit suitable for coadministration of the compositions.

Thus the kit of the invention comprises two or more separate pharmaceutical compositions, at least one of which contains a compound of Formula I, I-A, I-B, I-C, I-D, I-E, I-F and/or I-G in accordance with the invention, and means for separately retaining said compositions, such as a container, divided bottle, or divided foil packet. An example of such a kit is the familiar blister pack used for the packaging of tablets, capsules and the like.

### Methods of Use

It will be appreciated by those skilled in the art that references herein to treatment refer to the treatment of established conditions. However, the compounds of Formula I, I-A, I-B, I-C, I-D, I-E, I-F and/or I-G and pharmaceutically acceptable salts thereof may, depending on the condition, also be useful in the prevention (prophylaxis) of certain diseases (e.g. cryptosporidiosis).

As used herein, unless otherwise indicated, “treat”, “treating” or “treatment” in reference to a disease means: (1) to ameliorate the disease or one or more of the biological manifestations of the disease (2) to interfere with (a) one or more points in the biological cascade that leads to or is responsible for the disease or (b) one or more of the biological manifestations of the disease, (3) to alleviate one or more of the symptoms or effects associated with the disease, (4) to slow the progression of the disease or one or more of the biological manifestations of the disease, and/or (5) to diminish the likelihood of severity of a disease or biological manifestations of the disease.

As used herein, unless otherwise indicated, “prevent”, “preventing” or “prevention” means the prophylactic administration of a drug to diminish the likelihood of the onset of or to delay the onset of a disease or biological manifestation thereof. The skilled artisan will appreciate that “prevention” is not an absolute term. In medicine, “prevention” is understood to refer to the prophylactic administration of a drug to substantially diminish the likelihood or severity of a disorder or biological manifestation thereof, or to delay the onset of such disorder or biological manifestation thereof.

Thus, another aspect there is provided the treatment or prevention of a disease. In another aspect, there is provided the treatment of a disease. In a further aspect, there is provided the prevention of a disease.

There is thus provided as a further aspect of the invention a compound of Formula I, I-A, I-B, I-C, I-D, I-E, I-F and/or I-G, or a pharmaceutically acceptable salt thereof, for use in therapy. There is further provided a compound of Formula I, I-A, I-B, I-C, I-D, I-E, I-F and/or I-G, or a pharmaceutically acceptable salt thereof, for use as a medicament in therapy, which therapy is human or veterinary.

It will be appreciated that, when a compound Formula I, I-A, I-B, I-C, I-D, I-E, I-F and/or I-G or a pharmaceutically acceptable salt thereof is used in therapy, it is used as an active therapeutic agent.

For the avoidance of doubt, general references herein to “treatment” include references to curative, palliative and prophylactic treatment.

### Examples

The invention will now be illustrated by way of the following non-limiting examples. While particular embodiments of the invention are described below a skilled person will appreciate that various changes and modifications can be made. References to preparations carried out in a similar manner to, or by the general method of, other preparations, may encompass variations in routine parameters such as time, temperature, workup conditions, minor changes in reagents amounts, etc.

In certain of the following Intermediates and Examples, starting materials are identified by reference to other Intermediate or Example numbers. This does not signify that the actual material (or “batch”) obtained from any particular Intermediate or Example was necessarily used in a subsequent step exemplified herein, but is used as a short-hand means of denoting the relevant compound name.

The invention is illustrated by the following non-limiting examples in which the following abbreviations and definitions are used:

### ABBREVIATIONS

δ chemical shift
d doublet
dd double doublet
ACN acetonitrile
BID Bis in die
DCM dichloromethane
DMF dimethylformamide
ES low resolution electro spray mass spectroscopy
Et₃N triethylamine
EtOAc ethyl acetate
Eq(s) equivalent(s)
HPLC high performance liquid chromatography
HRMS high resolution mass spectrum
LCMS liquid chromatography mass spectrometry
m multiplet
min minutes
m/z mass spectrum peak
NMR nuclear magnetic resonance
PO per oral
q quartet
rt room temperature
S singlet
SFC supercritical fluid chromatography
t triplet
TBME Tertbutyl methyl ether
THE tetrahydrofuran
TLC thin layer chromatography

### Chemistry General Information: Equipment and Methods

Flash chromatography was performed using a Combiflash Companion Rf (commercially available from Teledyne ISCO) and prepacked silica gel columns purchased from Teledyne ISCO. Mass-directed preparative HPLC separations were performed using a Waters HPLC (2545 binary gradient pumps, 515 HPLC make up pump, 2767 sample manager) connected to a Waters 2998 photodiode array and a Waters 3100 mass detector. Preparative HPLC separations were performed with a Gilson HPLC (321 pumps, 819 injection module, 215 liquid handler/injector) connected to a Gilson 155 UV/vis detector. On both instruments, HPLC chromatographic separations were conducted using Waters XBridge C18 columns, 19×100 mm, 5 um particle size; using 0.1% ammonia in water (solvent A) and acetonitrile (solvent B) or 0.1% formic acid in water (solvent A) and acetonitrile (solvent B) as mobile phase. SFC chiral separation was performed on a Waters SFC 350. ¹H-NMR spectra were recorded on a Bruker Avance DPX 500 spectrometer (¹H at 500.1 MHz, ¹³C at 125 MHz ¹⁹F at 470.5 MHz), BrukerAvance DPX 400 (1H at 400 MHz) or a Bruker Avance DPX 400 (1H at 300 MHz). Chemical shifts (6) are expressed in p.p.m recorded using the residual solvent as the internal reference in all cases. Signal splitting patterns are described as singlet (s), doublet (d), triplet (t), quartet (q), multiplet (m), broad (br), or a combination thereof. Coupling constants (J) are quoted to the nearest 0.1 Hz. Low resolution electrospray (ES) mass spectra were recorded on an Advion Compact Mass Spectrometer (CMS; model Expression CMS) connected to Dionex Ultimate 3000 UPLC system with diode array detector. HPLC chromatographic separations were conducted using a Waters XBridge C18 column, 2.1×50 mm, 3.5 μm particle size or Waters XSelect 2.1×30 mm, 2.5 μm particle size. The compounds were eluted with a gradient of 5 to 95% acetonitrile/water+0.1% Ammonia or +0.1% formic acid. High resolution mass spectroscopy (HRMS) was performed using a Bruker MicroTof mass spectrometer or Thermo Exploris 120 Orbitrap. Unless otherwise stated herein reactions have not been optimised. Solvents and reagents were purchased from commercial suppliers and used without further purification. Dry solvents were purchased in sure sealed bottles stored over molecular sieves.

### Synthesis

### General scheme for the synthesis of 2-amino-4-(trifluoromethyl)-6-(((1R,3S)-3-(trifluoromethyl)cyclohexyl)methyl)-6,7-dihydro-5H-pyrrolo[3,4-d]pyrimidin-5-one (Compound 1)

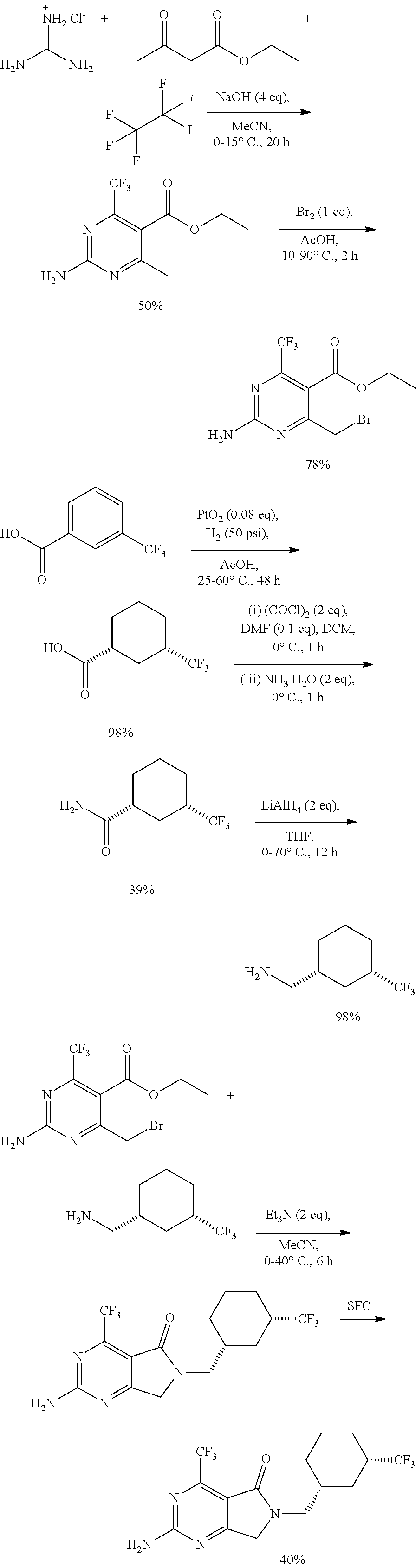

### ethyl 2-amino-4-methyl-6-(trifluoromethyl)pyrimidine-5-carboxylate (Intermediate 1)

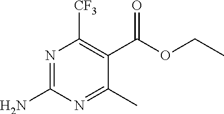 [SMILES: CCOC(=O)c1c(C)nc(N)nc1C(F)(F)F]

A mixture of guanidine hydrochloride (121 g, 1.27 mol) and NaOH (189 g, 4.73 mol) in MeCN (1.5 L) in a sealed tube was stirred at 0° C. for 5 h under nitrogen. Ethyl 3-oxobutanoate (150 g, 1.15 mol) and 1,1,1,2,2-pentafluoro-2-iodo-ethane (312 g, 1.27 mol) were added and the reaction mixture was stirred at 15° C. for 15 h. The reaction mixture was quenched with water (3 L) and extracted with ethyl acetate (3×2 L). The combined organics were washed with brine (1.5 L), dried over Na₂SO₄, filtered, and concentrated under vacuum. The product was purified by column chromatography (SiO₂, 0:100→50:50 EtOAc:petroleum ether) to afford ethyl 2-amino-4-methyl-6-(trifluoromethyl)pyrimidine-5-carboxylate (288 g, 50% yield) as a yellow solid. ¹H NMR (400 MHz, DMSO-d6) δ 7.64 (br. s, 2H), 4.28 (q, J=7.2 Hz, 2H), 2.35 (s, 3H), 1.27 (t, J=6.8 Hz, 3H). MS (ESI): m/z (%) 250 (80) [M+H]⁺.

### ethyl 2-amino-4-(bromomethyl)-6-(trifluoromethyl)pyrimidine-5-carboxylate (Intermediate 2)

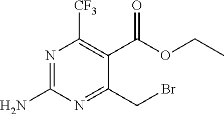 [SMILES: CCOC(=O)c1c(CBr)nc(N)nc1C(F)(F)F]

To a mixture of ethyl 2-amino-4-methyl-6-(trifluoromethyl) pyrimidine-5-carboxylate (180 g, 722 mmol) in AcOH (1.6 L) was added a solution of Br₂ (115 g, 722 mmol) in AcOH (200 mL) at 10° C. under nitrogen and the reaction mixture was heated at 90° C. for 2 h. The reaction mixture was concentrated then quenched with water (1 L) and extracted with ethyl acetate (3×800 mL). The combined organics were washed with brine (1 L), dried over Na₂SO₄, filtered, and concentrated under vacuum. The product was purified by column chromatography (SiO₂, 5:95→15:85 EtOAc:petroleum ether) to afford ethyl 2-amino-4-(bromomethyl)-6-(trifluoromethyl)pyrimidine-5-carboxylate (370 g, 78% yield) as a yellow solid.

¹H NMR (400 MHz, DMSO-d6) δ 7.95 (br. s, 2H), 4.50 (s, 2H), 4.30 (q, J=7.2 Hz, 2H), 1.28 (t, J=7.2 Hz, 3H). MS (ESI): m/z (%) 330/331 (97) [M+H]⁺.

### 3-(trifluoromethyl)cyclohexane-1-carboxylic acid (Intermediate 3)

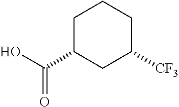 [SMILES: O=C(O)[C@@H]1CCC[C@H](C(F)(F)F)C1]

To a solution of 3-(trifluoromethyl)benzoic acid (100 g, 526 mmol) in AcOH (1 L) was added PtO₂ (10 g, 44 mmol). The suspension was degassed under vacuum, purged with H₂ and stirred under H₂ (50 psi) at 60° C. for 48 h. The reaction mixture was filtered, concentrated under vacuum, and azeotroped with toluene to afford racemic cis 3-(trifluoromethyl) cyclohexane-1-carboxylic acid (405 g, 98% yield) as a colourless oil. ¹H NMR (400 MHz, DMSO-d6) δ 12.20 (br. s, 1H), 2.47-2.26 (m, 2H), 2.16-1.92 (m, 2H), 1.87-1.74 (m, 2H), 1.51-1.08 (m, 4H).

### 3-(trifluoromethyl)cyclohexane-1-carboxamide (Intermediate 4)

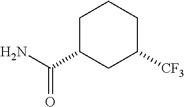 [SMILES: NC(=O)[C@@H]1CCC[C@H](C(F)(F)F)C1]

To a solution of racemic cis 3-(trifluoromethyl)cyclohexanecarboxylic acid (260 g, 1.33 mol) in DCM (2.5 L) at 0° C. was added (COCl)₂ (232 mL, 2.65 mol), followed by DMF (10.2 mL, 132 mmol) and the reaction mixture was stirred at 0° C. for 1 h. The reaction mixture was concentrated under vacuum then dissolved in DCM (500 mL) and added to NH₃·H₂O (408 mL, 2.65 mol) at 0° C. The reaction mixture was stirred at 0° C. for 1 h then concentrated under vacuum and azeotroped with toluene. The product was purified by column chromatography (SiO₂, 30:70→50:50 EtOAc:petroleum ether) to afford racemic cis 3-(trifluoromethyl)cyclohexanecarboxamide (200 g, 39% yield) as a white solid. ¹H NMR (400 MHz, DMSO-d6) δ 7.27 (s, 1H), 6.77 (s, 1H), 2.38-2.08 (m, 2H), 1.98-1.67 (m, 4H), 1.41-1.06 (m, 4H).

### 3-(trifluoromethyl)cyclohexyl)methanamine (Intermediate 5)

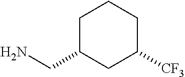 [SMILES: NC[C@@H]1CCC[C@H](C(F)(F)F)C1]

LiAlH₄ (32 g, 843 mmol) was slowly added into THE (600 mL) at 0° C. under nitrogen. A solution of racemic cis 3-(trifluoromethyl) cyclohexanecarboxamide (82 g, 420 mmol) in THE (600 mL) at 0° C. was added dropwise over 30 min under nitrogen. The reaction mixture was heated at 70° C. for 12 h then cooled to 0° C. and quenched by dropwise addition of water (32 mL). 3.75 M NaOH (32 mL) and water (96 mL) were added and the mixture was filtered and concentrated under vacuum to afford racemic cis 3-(trifluoromethyl)cyclohexyl)methanamine (150 g, 98% yield) as a yellow oil. ¹H NMR (400 MHz, DMSO-d6) δ 2.47-2.33 (m, 2H), 2.29-2.12 (m, 1H), 1.93 (br. d, J=12.4 Hz, 1H), 1.87-1.67 (m, 4H), 1.34-1.04 (m, 4H), 0.90-0.71 (m, 2H).

### 2-amino-4-(trifluoromethyl)-6-(((1R,3S)-3-(trifluoromethyl)cyclohexyl)methyl)-6,7-dihydro-5H-pyrrolo[3,4-d]pyrimidin-5-one (Compound 1)

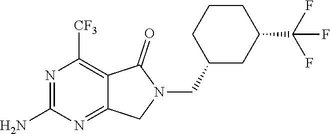 [SMILES: Nc1nc2c(c(C(F)(F)F)n1)C(=O)N(C[C@@H]1CCC[C@H](C(F)(F)F)C1)C2]

To a mixture of racemic cis 3-(trifluoromethyl)cyclohexyl)methanamine (80 g, 442 mmol) and Et₃N (82 mL, 589 mmol) in MeCN (800 mL) at 0° C. under nitrogen was added ethyl 2-amino-4-(bromomethyl)-6-(trifluoromethyl)pyrimidine-5-carboxylate (97 g, 294 mmol) in MeCN (200 mL). The reaction mixture was stirred at 0° C. for 2 h, then heated to 40° C. for 4 h. The reaction mixture was concentrated under vacuum then quenched with water (800 mL) and extracted with ethyl acetate (3×800 mL). The combined organics were washed with brine (700 mL), dried over Na₂SO₄, filtered, and concentrated under vacuum. The product was purified by column chromatography (SiO₂, 15:85→40:60 EtOAc:petroleum ether) then the enantiomers were separated by chiral SFC (column: Daicel Chiralpak IG (250 mm*50 mm, 10 um); mobile phase: [Hexane—EtOH]; B %: 15%-15%, 3.9; 6100 min) to afford 2-amino-4-(trifluoromethyl)-6-(((1R,3S)-3-(trifluoromethyl)cyclohexyl)methyl)-6,7-dihydro-5H-pyrrolo [3,4-d]pyrimidin-5-one (81 g, 40% yield) as a yellow solid. ¹H NMR (400 MHz, DMSO-d6) δ 8.11-7.77 (m, 2H), 4.48-4.32 (m, 2H), 3.41-3.23 (m, 2H), 2.29-2.16 (m, 1H), 1.86-1.73 (m, 4H), 1.65 (br. d, J=12.4 Hz, 1H), 1.35-1.09 (m, 2H), 0.99-0.77 (m, 2H). ¹³C NMR (125 MHz, DMSO-d6) δ 176.4, 163.8, 162.6, 150.8 (q, ²J_{CF}=37.9 Hz), 128.0 (q, ¹J_{CF}=278.5 Hz), 119.9 (q, ¹J_{CF}=279.5 Hz), 117.9, 109.0, 51.2, 47.5, 34.8, 29.3, 28.6, 24.4, 23.6. ¹⁹F NMR (376 MHz, DMSO-d6) δ −66.22, −72.48. HRMS (ESI) calcd. for [M+H]⁺ C₁₅H₁₇F₆N₄O, 383.1307, found 383.1305.

### General scheme for the synthesis of 2-amino-4-methyl-6-(((1R,3S)-3-(trifluoromethyl)cyclohexyl)methyl)-6,7-dihydro-5H-pyrrolo[3,4-d]pyrimidin-5-one (Compound 2)

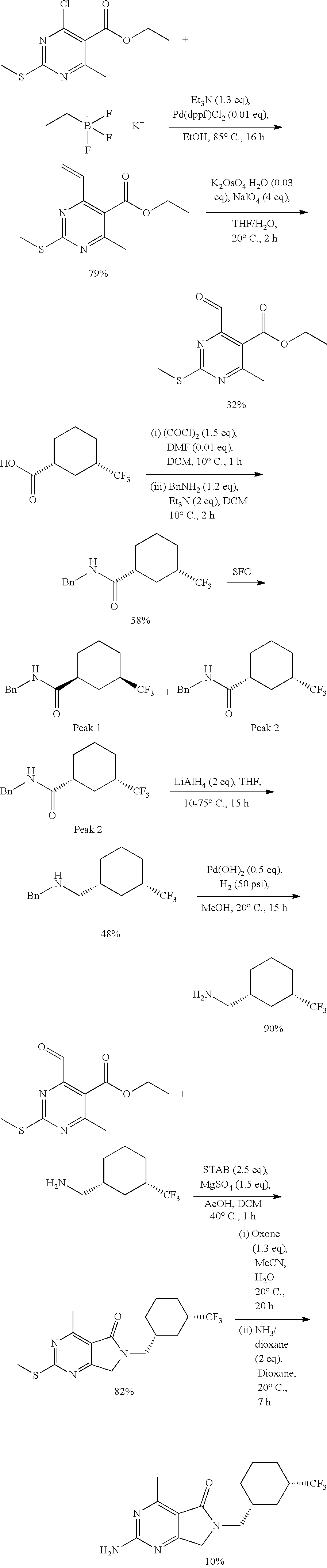

### ethyl 4-methyl-2-(methylthio)-6-vinylpyrimidine-5-carboxylate (Intermediate 6)

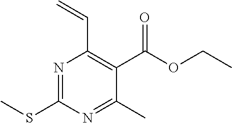 [SMILES: C=Cc1nc(SC)nc(C)c1C(=O)OCC]

To a solution of ethyl 4-chloro-6-methyl-2-(methylthio)pyrimidine-5-carboxylate (7.7 g, 31.2 mmol) in ethanol (70 mL) was added potassium vinyltrifluoroborate (5.02 g, 37.5 mmol), Et₃N (5.64 mL, 40.6 mmol) and Pd(dppf)Cl₂ (255 mg, 0.31 mmol) and the reaction mixture was purged with nitrogen and heated at 85° C. under nitrogen for 16 h. The reaction mixture was concentrated under vacuum then diluted with water (40 mL) and extracted with EtOAc (3×40 mL). The combined organics were washed with brine (40 mL), dried over MgSO₄, filtered, and concentrated under vacuum. The product was purified by column chromatography (80 g silica, 0:100→30:70 EtOAc:heptane) to afford ethyl 4-methyl-2-methylsulfanyl-6-vinyl-pyrimidine-5-carboxylate (6.15 g, 79% yield) as a colourless oil. ¹H NMR (400 MHz, CDCl₃): δ 6.83 (dd, J=16.8, 10.3 Hz, 1H), 6.70 (dd, J=16.8, 2.1 Hz, 1H), 5.67 (dd, J=10.3, 2.1 Hz, 1H), 4.41 (q, J=7.1 Hz, 2H), 2.59 (s, 3H), 2.49 (s, 3H), 1.39 (t, J=7.1 Hz, 3H). MS (ES+): m/z (%) 239 (90) [M+H]⁺.

### ethyl 4-formyl-6-methyl-2-(methylthio)pyrimidine-5-carboxylate (Intermediate 7)

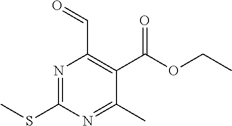 [SMILES: CCOC(=O)c1c(C)nc(SC)nc1C=O]

To a solution of ethyl 4-methyl-2-methylsulfanyl-6-vinyl-pyrimidine-5-carboxylate (6.02 g, 25.3 mmol) in THE (40 mL) and water (10 mL) was added a solution of dipotassium dioxido(dioxo)osmium dihydrate (326 mg, 0.88 mmol) in water (10 mL). The reaction mixture was stirred at room temperature for 15 min, then a suspension of sodium (meta)periodate (21.6 g, 101 mmol) in water (10 mL) was added dropwise and the reaction mixture was stirred at room temperature for 2 h. The reaction mixture was diluted with water (20 mL) and extracted with EtOAc (3×20 mL). The combined organics were washed with brine (20 mL) and passed through a hydrophobic frit. Residual osmium was quenched by the addition of corn oil (1 mL) and the organics were concentrated under vacuum. The product was purified by column chromatography (40 g silica, 0:100→60:40 EtOAc:heptane) to afford ethyl 4-formyl-6-methyl-2-methylsulfanyl-pyrimidine-5-carboxylate (1.93 g, 32% yield) as a pale brown oil. ¹H NMR (400 MHz, CDCl₃): δ 9.94 (s, 1H), 4.45 (q, J=5.7 Hz, 2H), 2.64 (s, 3H), 2.58 (s, 3H), 1.39 (t, J=5.7 Hz, 3H). MS (ES+): m/z (%) 241 (94) [M+H]⁺.

### N-benzyl-3-(trifluoromethyl)cyclohexane-1-carboxamide (Intermediate 8)

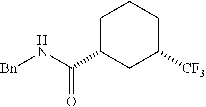 [SMILES: O=C(NCc1ccccc1)[C@@H]1CCC[C@H](C(F)(F)F)C1]

To a solution of racemic cis3-(trifluoromethyl)cyclohexanecarboxylic acid (51 g, 259 mmol) in DCM (510 mL) at 10° C. was added (COCl)₂ (34 mL, 389 mmol), followed by DMF (0.2 mL, 2.6 mmol) and the reaction mixture was stirred at 10° C. for 1 h. The reaction mixture was concentrated under vacuum, azeotroped with toluene then dissolved in DCM (600 mL). Benzylamine (33.5 mL, 307 mmol) and Et₃N (71 mL, 512 mmol) were added and the reaction mixture was stirred at 10° C. for 2 h. The reaction mixture was poured into water (1.5 L) and extracted with DCM (3×300 mL). The combined organics were washed with 0.5 M HCl (200 mL), aq. NaHCO₃ (200 mL), and brine (200 mL), then dried over Na₂SO₄, filtered and concentrated under vacuum. The product was purified by column chromatography (SiO₂, 15:85→25:75 EtOAc:petroleum ether) to afford racemic cis N-benzyl-3-(trifluoromethyl)cyclohexane-1-carboxamide (45 g, 58% yield) as a white solid. ¹H NMR (400 MHz, CDCl₃) δ 7.28-7.19 (m, 5H), 5.71 (br. s, 1H), 4.37 (d, J=6.0 Hz, 2H), 2.11-2.04 (m, 3H), 1.90-1.88 (m, 3H), 1.56-1.48 (m, 2H), 1.24-1.23 (m, 2H). MS (ESI): m/z (%) 285 (95) [M+H]⁺.

### (1R,3S)-N-benzyl-3-(trifluoromethyl)cyclohexane-1-carboxamide (Intermediate 9)

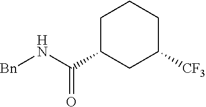 [SMILES: O=C(NCc1ccccc1)[C@@H]1CCC[C@H](C(F)(F)F)C1]

Racemic cis N-benzyl-3-(trifluoromethyl)cyclohexane-1-carboxamide was purified by SFC (column: Daicel Chiralpak AY (250 mm*50 mm, 10 um); mobile phase: [0.1% NH₃H₂O MEOH]; B %: 55%-55%, 2.6 min; 360 min) to afford (1R,3S)-N-benzyl-3-(trifluoromethyl)cyclohexane-1-carboxamide (peak 2, 5.8 g, 48% yield) as a white solid. ¹H NMR (400 MHz, CDCl₃) δ 7.35-7.23 (m, 5H), 5.73 (br. s, 1H), 4.42 (d, J=5.6 Hz, 2H), 2.18-1.99 (m, 3H), 1.99-1.85 (m, 3H), 1.60-1.39 (m, 2H), 1.36-1.20 (m, 2H). MS (ESI): m/z (%) 285 (99) [M+H]⁺.

### N-benzyl-1-((1R,3S)-3-(trifluoromethyl)cyclohexyl)methanamine (Intermediate 10)

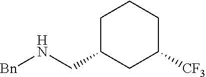 [SMILES: FC(F)(F)[C@H]1CCC[C@@H](CNCc2ccccc2)C1]

To a mixture of (1R,3S)-N-benzyl-3-(trifluoromethyl)cyclohexane-1-carboxamide (5.8 g, 20 mmol) in THE (60 mL) at 10° C. was added LiAlH₄ (1.5 g, 41 mmol) and the reaction mixture was heated at 75° C. for 15 h. The reaction mixture was quenched with water (1.5 mL). 3.75 M NaOH (1.5 mL) and water (4.6 mL), then Na₂SO₄ (16 g) were added and the mixture was filtered and concentrated under vacuum. The crude product was purified by HPLC (column: Phenomenex luna C18 150*40 mm* 15 um; mobile phase: [water (0.1% TFA)-ACN]; B %:17%-47%, 10 min) to afford N-benzyl-1-((1R,3S)-3-(trifluoromethyl)cyclohexyl)methanamine (2.85 g, 48% yield) as a yellow oil. ¹H NMR (400 MHz, CDCl₃) δ 7.30-7.17 (m, 5H), 3.73 (s, 2H), 2.52-2.40 (m, 2H), 2.05-1.91 (m, 2H), 1.90-1.72 (m, 3H), 1.55-1.40 (m, 1H), 1.30-1.08 (m, 2H), 0.96-0.74 (m, 2H). MS (ESI): m/z (%) 271 (93) [M+H]⁺.

### ((1R,3S)-3-(trifluoromethyl)cyclohexyl)methanamine (Intermediate 11)

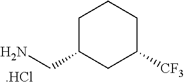 [SMILES: NC[C@@H]1CCC[C@H](C(F)(F)F)C1 ; ".HCl"]

To a solution of N-benzyl-1-((1R,3S)-3-(trifluoromethyl)cyclohexyl)methanamine (2.85 g, 10.5 mmol) in MeOH (28 mL) was added Pd(OH)₂ (737 mg, 5.25 mmol) under N₂. The suspension was degassed under vacuum, purged with H₂ and stirred under H₂ (50 psi) at 20° C. for 15 h. The reaction mixture was filtered. HCl/MeOH (50 mL) was added, and the filtrate was concentrated under vacuum. The product was triturated with EtOAc/petroleum ether (10:90) then filtered and dried under vacuum to afford ((1R,3S)-3-(trifluoromethyl)cyclohexyl)methanamine (2.06 g, 90% yield) as a pale yellow solid. ¹H NMR (400 MHz, DMDO-d₆) δ 7.99 (s, 3H), 2.77-2.61 (m, 2H), 2.32-2.27 (m, 1H), 1.96 (d, J=12.4 Hz, 1H), 1.88-1.71 (m, 3H), 1.71-1.58 (m, 1H), 1.37-1.21 (m, 1H), 1.21-1.06 (m, 1H), 1.01-0.81 (m, 2H).

### 4-methyl-2-(methylthio)-6-(((1R,3S)-3-(trifluoromethyl)cyclohexyl)methyl)-6,7-dihydro-5H-pyrrolo[3,4-d]pyrimidin-5-one (Intermediate 12)

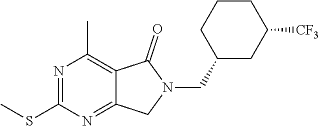 [SMILES: CSc1nc(C)c2c(n1)CN(C[C@@H]1CCC[C@H](C(F)(F)F)C1)C2=O]

To a solution of ethyl 4-formyl-6-methyl-2-methylsulfanyl-pyrimidine-5-carboxylate (1.93 g, 8.03 mmol) in DCM (40 mL) was added (1R,3S))-(3-(trifluoromethyl)cyclohexyl]methanamine (2.33 g, 12.9 mmol), acetic acid (20 μL) and MgSO₄ (1.45 g, 12.0 mmol). The reaction mixture was stirred at room temperature for 10 min under nitrogen. Sodium triacetoxyborohydride (4.26 g, 20.1 mmol) was added portionwise and the reaction mixture was heated at 40° C. for 1 h. The reaction mixture was diluted with DCM (20 mL), washed with water (3×20 mL), passed through a hydrophobic frit, and concentrated under vacuum. The product was purified by column chromatography (80 g silica, 0:100→60:40 EtOAc:heptane) to afford 4-methyl-2-(methylthio)-6-(((1R,3S)-3-(trifluoromethyl) cyclohexyl)methyl)-6,7-dihydro-5H-pyrrolo[3,4-d]pyrimidin-5-one (2.38 g, 83% yield) as a pale gold oil. ¹H NMR (400 MHz, CDCl₃): δ 4.32 (s, 2H), 3.50 (dd, J=13.9, 7.3 Hz, 1H), 3.43 (dd, J=13.9, 6.9 Hz, 1H), 2.79 (s, 3H), 2.62 (s, 3H), 2.08-2.00 (m, 1H), 1.97-1.90 (m, 2H), 1.84-1.75 (m, 2H), 1.33-1.23 (m, 1H), 1.13-1.00 (m, 2H), 0.89 (t, J=6.9 Hz, 2H). MS (ES+): m/z (%) 360 (98) [M+H]⁺.

### 2-amino-4-methyl-6-(((1R,3S)-3-(trifluoromethyl)cyclohexyl)methyl)-6,7-dihydro-5H-pyrrolo[3,4-d]pyrimidin-5-one (Compound 2)

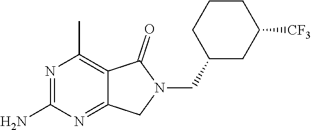 [SMILES: Cc1nc(N)nc2c1C(=O)N(C[C@@H]1CCC[C@H](C(F)(F)F)C1)C2]

To a solution of 4-methyl-2-methylsulfanyl-6-[[3-(-1R,3S)-(trifluoromethyl)cyclohexyl]methyl]-7H-pyrrolo[3,4-d]pyrimidin-5-one (2.38 g, 6.62 mmol) in MeCN (20 mL) was added a solution of oxone (5.29 g, 8.61 mmol) in water (10 mL) and the reaction mixture was stirred at room temperature for 20 h. The reaction mixture was diluted with water (10 mL), extracted with DCM (3×10 mL), passed through a hydrophobic frit, and concentrated under vacuum. The residue was dissolved in 1,4-dioxane (10 mL) and a solution of ammonia in dioxane (26.5 mL, 13.2 mmol, 0.5 M) was added slowly. The reaction mixture was stirred at room temperature for 7 h then concentrated under vacuum. The product was recrystallised from EtOH to afford 2-amino-4-methyl-6-(((1R,3S)-3-(trifluoromethyl) cyclohexyl)methyl)-6,7-dihydro-5H-pyrrolo[3,4-d]pyrimidin-5-one (231 mg, 10% yield) as an off-white solid. ¹H NMR (400 MHz, DMSO-d6): δ 7.17 (br. s, 2H), 4.29-4.18 (m, 2H), 3.36-3.31 (m, 1H), 3.27-3.23 (m, 1H), 2.47 (s, 3H), 2.31-2.20 (m, 1H), 1.84-1.74 (m, 4H), 1.63 (br. d, J=12.7 Hz, 1H), 1.30-1.11 (m, 2H), 0.96-0.86 (m, 2H). ¹³C NMR (100 MHz, DMSO-d6): δ 173.3, 166.3, 165.3, 164.1, 127.9 (q, J_{CF}=250.4 Hz), 110.8, 50.6, 47.1, 34.9, 29.3, 28.6, 24.4, 23.6, 19.6. ¹⁹F NMR (470 MHz, DMSO-d6) δ −72.34. HRMS (ES+) calcd. for C₁₅H₂₀ON₄F₃ [M+H]⁺329.1589, found 329.1584.

### General scheme for the synthesis of 2-amino-4-(difluoromethyl)-6-(((1R,3S)-3-(trifluoromethyl)cyclohexyl)methyl)-6,7-dihydro-5H-pyrrolo[3,4-d]pyrimidin-5-one (Compound 3)

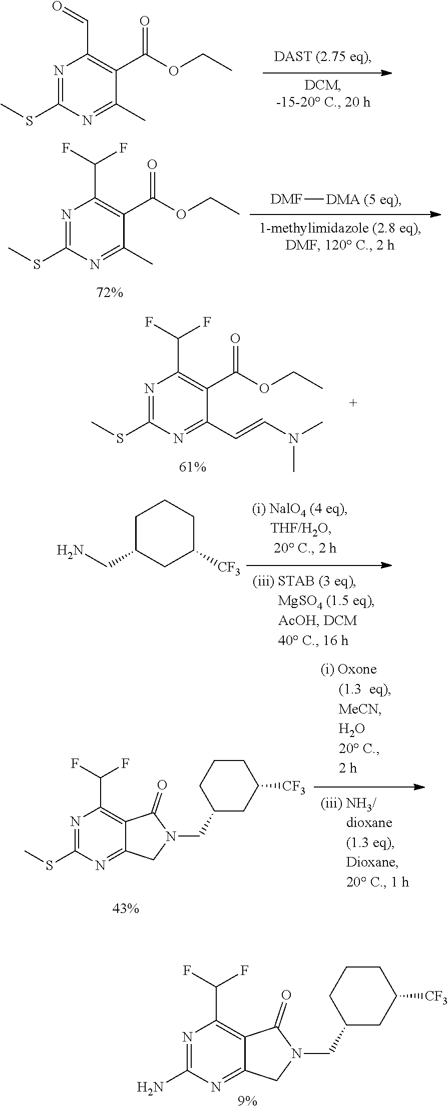

### ethyl 4-(difluoromethyl)-6-methyl-2-(methylthio)pyrimidine-5-carboxylate (Intermediate 13)

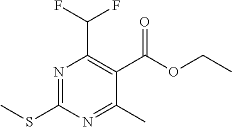 [SMILES: CCOC(=O)c1c(C)nc(SC)nc1C(F)F]

A solution of ethyl 4-formyl-6-methyl-2-methylsulfanyl-pyrimidine-5-carboxylate (1.08 g, 4.50 mmol) in DCM (12 mL) with stirring under nitrogen was cooled to −15° C. (Diethylamino)sulfur trifluoride (1.99 g, 12.4 mmol) was added slowly over 10 min and the reaction mixture was allowed to warm to room temperature over 2 h then stirred at room temperature for 18 h. Ice-water (20 mL) and DCM (25 mL) were added, and the two-phase system was shaken and separated. The aqueous layer was extracted with DCM (3×25 mL) and the combined organics were dried (MgSO₄), filtered, and concentrated under vacuum. The product was purified by column chromatography (12 g silica, 0:100→30:70 EtOAc:heptane to afford ethyl 4-(difluoromethyl)-6-methyl-2-methylsulfanyl-pyrimidine-5-carboxylate (870 mg, 72% yield) as a pale thick gold oil. ¹H NMR (500 MHz, CDCl₃) δ 6.68 (t, J=54.3 Hz, 1H), 4.42 (q, J=7.1 Hz, 2H), 2.60 (s, 3H), 2.59 (s, 3H), 1.39 (t, J=7.2 Hz, 3H). MS (ES+): m/z (%) 263.1 (99) [M+H]⁺.

### ethyl (E)-4-(difluoromethyl)-6-(2-(dimethylamino)vinyl)-2-(methylthio)pyrimidine-5-carboxylate (Intermediate 14)

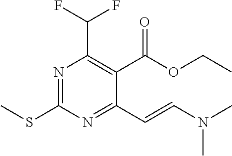

To a solution of ethyl 4-(difluoromethyl)-6-methyl-2-methylsulfanyl-pyrimidine-5-carboxylate (770 mg, 2.94 mmol) in DMF (10 mL) under nitrogen was added (dimethylamino)acetaldehyde dimethyl acetal (1.95 mL, 14.7 mmol) followed by 1-methylimidazole (0.65 mL, 8.22 mmol) and the reaction mixture was heated to 120° C. for 2 h. The reaction mixture was allowed to cool to room temperature, then diluted with water (10 mL) and extracted with EtOAc-TBME (5:1) (3×15 mL). The combined organics were washed with brine (15 mL), dried (MgSO₄), filtered, and concentrated under vacuum. The product was purified by column chromatography (12 g silica, 0:100→50:50 EtOAc:heptane to afford ethyl 4-(difluoromethyl)-6-[(E)-2-(dimethylamino)vinyl]-2-methylsulfanyl-pyrimidine-5-carboxylate (595 mg, 61% yield) as a pale yellow powder. ¹H NMR (500 MHz, CDCl₃) δ 8.08 (d, J=12.3 Hz, 1H), 6.56 (t, J=54.6 Hz, 1H), 5.30 (d, J=12.3 Hz, 1H), 4.38 (q, J=7.1 Hz, 2H), 3.00 (br s, 6H), 2.55 (s, 3H), 1.38 (t, J=7.1 Hz, 3H). MS (ES+): m/z (%) 318.1 (79) [M+H]⁺+m/z (%) 291.0 (12) [M-(N(CH₃)₂)+OH+H]⁺.

### 4-(difluoromethyl)-2-(methylthio)-6-(((1R,3S)-3-(trifluoromethyl)cyclohexyl)methyl)-6,7-dihydro-5H-pyrrolo[3,4-d]pyrimidin-5-one (Intermediate 15)

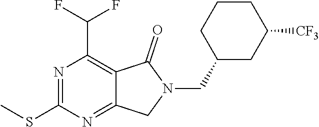 [SMILES: CSc1nc2c(c(C(F)F)n1)C(=O)N(C[C@@H]1CCC[C@H](C(F)(F)F)C1)C2]

To a solution of sodium (meta)periodate (5.66 g, 26.5 mmol) in water (20 mL) at 0-5° C. was added a solution of ethyl 4-(difluoromethyl)-6-[(E)-2-(dimethylamino)vinyl]-2-methylsulfanyl-pyrimidine-5-carboxylate (2.1 g, 6.62 mmol) in THE (35 mL) dropwise over 10 min (exotherm kept below 10° C.). The reaction mixture was stirred at room temperature for 2 h then diluted with DCM (30 mL). The aqueous layer was separated and extracted with DCM (2×15 mL). The combined organics were washed with brine (20 mL), dried (MgSO₄), filtered, and concentrated under vacuum to a volume of 30 mL. To this solution at room temperature under nitrogen was added (1R,3S)-3-(trifluoromethyl)cyclohexyl)methanamine (1.44 g, 7.94 mmol), acetic acid (0.05 mL) and MgSO₄ (1.19 g, 9.93 mmol). The reaction mixture was stirred at room temperature for 10 min, then sodium (triacetoxy)borohydride (4.21 g, 19.9 mmol) was added portionwise. The reaction mixture was heated at 40° C. for 18 h then cooled to room temperature. Further portions of (1R,3S)-3-(trifluoromethyl)cyclohexyl)methanamine (720 mg, 3.97 mmol) and MgSO₄ (597 mg, 4.96 mmol) were added and the reaction mixture was stirred at room temperature for 20 min. Sodium (triacetoxy)borohydride (2.1 g, 9.92 mmol) was added and the reaction mixture was heated at 40° C. for 2 h then cooled to room temperature. The reaction mixture was diluted with DCM (20 mL), washed with water (10 mL), passed through a hydrophobic frit, dried (MgSO₄), filtered, and concentrated under vacuum. The product was purified by column chromatography (40 g silica, 0:100→50:50 EtOAc:heptane) to afford an orange-brown oily solid. The solid was sonicated in TBME (1.7 mL), allowed to stand at rt for 30 min, then the supernatant was removed by pipette. This sonication procedure was repeated, and the solid obtained was dried to give a yellow powder. The supernatant was concentrated and then purified by column chromatography (12 g silica, 0:100→50:50 EtOAc:heptane). The purified fractions were combined with the previously purified solid and concentrated under vacuum. The resulting solid was sonicated in TBME (1.3 mL), the supernatant removed and the solid dried to afford 4-(difluoromethyl)-2-(methylthio)-6-(((1R,3S)-3-(trifluoromethyl)cyclohexyl)methyl)-6,7-dihydro-5H-pyrrolo[3,4-d]pyrimidin-5-one (1.19 g, 43% yield) as a yellow powder. ¹H NMR (400 MHz, CDCl₃) δ 7.19 (t, J=26.8 Hz, 1H), 4.37 (s, 2H), 3.50-3.35 (m, 2H), 2.60 (s, 3H), 2.06-1.64 (m, 6H), 1.33-1.10 (m, 2H), 1.10-0.88 (m, 2H). MS (ES+): m/z (%) 396.0 (86) [M+H]⁺.

### 2-amino-4-(difluoromethyl)-6-(((1R,3S)-3-(trifluoromethyl)cyclohexyl)methyl)-6,7-dihydro-5H-pyrrolo[3,4-d]pyrimidin-5-one (Compound 3)

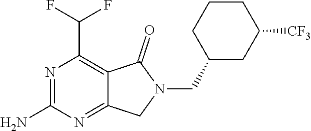 [SMILES: Nc1nc2c(c(C(F)F)n1)C(=O)N(C[C@@H]1CCC[C@H](C(F)(F)F)C1)C2]

To a solution of 4-(difluoromethyl)-2-(methylthio)-6-(((1R,3S)-3-(trifluoromethyl)cyclohexyl)methyl)-6,7-dihydro-5H-pyrrolo[3,4-d]pyrimidin-5-one (1.19 g, 3.01 mmol) in MeCN (20 mL) was added a solution of oxone (2.41 g, 3.91 mmol) in water (10 mL) and the reaction mixture was stirred at room temperature for 18 h. The reaction mixture was diluted with water (20 mL), extracted with DCM (3×20 mL), and passed through a hydrophobic frit. The combined organics were washed with brine (20 mL), dried, and concentrated under vacuum. The residue was dissolved in 1,4-dioxane (15 mL) and cooled to 0-5° C. A cooled 0.5 M solution of ammonia (7.83 mL, 3.91 mmol) in 1,4-dioxane was added dropwise and the reaction mixture was stirred at room temperature for 1 h. The reaction mixture was concentrated under vacuum and the product was purified by column chromatography (80 g silica, 0:100→80:20 EtOAc:heptane). The resulting solid was sonicated in EtOH (5 mL), allowed to stand for 30 min then filtered. This was repeated twice and the supernatants were combined and concentrated under vacuum to give a solid which was purified by column chromatography (4 g silica, 0:100→80:20 EtOAc:heptane). The resulting solid was purified by preparative HPLC (20:80→95:5 MeCN:water, 0.1% ammonia modifier) then sonicated in MeCN (0.8 mL) with two drops of MeOH, allowed to stand at room temperature for 30 mins, filtered, and dried to afford 2-amino-4-(difluoromethyl)-6-(((1R,3S)-3-(trifluoromethyl)cyclohexyl)methyl)-6,7-dihydro-5H-pyrrolo[3,4-d]pyrimidin-5-one (96 mg, 9% yield) as a pale pink powder. ¹H NMR (500 MHz, DMSO-d6) δ 8.00-7.61 (m, 2H), 7.27 (t, J=53.8 Hz, 1H), 4.46-4.33, (m, 2H), 3.33-3.25 (m, 2H), 2.32-2.16 (m, 1H), 1.87-1.72 (m, 4H), 1.70-1.59 (m, 1H), 1.34-1.21 (m, 1H), 1.21-1.09 (m, 1H), 0.99-0.84 (m, 2H). ¹³C NMR (400 MHz, DMSO-d6) δ 174.9, 164.6, 164.3, 156.1 (t, ¹J_{CF}=22.8 Hz), 127.9 (q, ¹J_{CF}=278.5 Hz), 108.7, 106.2, 51.4, 47.3, 40.2, 34.8, 29.3, 28.6, 24.3, 23.6. ¹⁹F NMR (500 MHz, DMSO-d₆) −72.3, −122.0. HRMS (ES+) calcd. for C₁₅H₁₈F₅N₄O [M+H]⁺365.1323, found 365.1402.

### General scheme for the synthesis of 2-amino-6-(spiro[2.5]octan-5-ylmethyl)-4-(trifluoromethyl)-6,7-dihydro-5H-pyrrolo[3,4-d]pyrimidin-5-one (Compound 4)

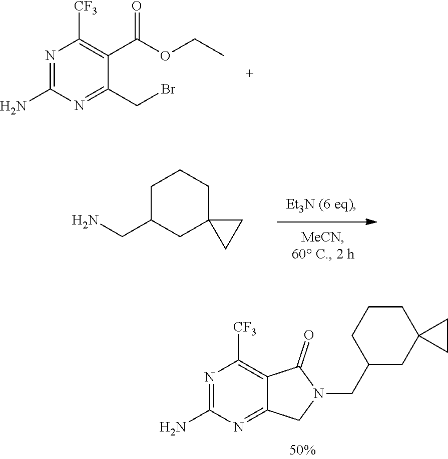 [SMILES: CCOC(=O)c1c(CBr)nc(N)nc1C(F)(F)F ; NCC1CCCC2(CC2)C1 ; Nc1nc2c(c(C(F)(F)F)n1)C(=O)N(CC1CCCC3(CC3)C1)C2]

### 2-amino-6-(spiro[2.5]octan-5-ylmethyl)-4-(trifluoromethyl)-6,7-dihydro-5H-pyrrolo[3,4-d]pyrimidin-5-one (Compound 4)

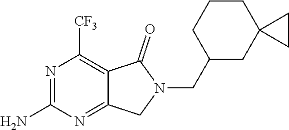 [SMILES: Nc1nc2c(c(C(F)(F)F)n1)C(=O)N(CC1CCCC3(CC3)C1)C2]

To a solution of spiro[2.5]octan-7-ylmethanamine hydrochloride (375 mg, 2.13 mmol) and Et₃N (0.89 mL, 6.4 mmol) in MeCN (2 mL) was added ethyl 2-amino-4-(bromomethyl)-6-(trifluoromethyl) pyrimidine-5-carboxylate (350 mg, 1.07 mmol) and the reaction mixture was stirred at room temperature for 30 min then heated at 60° C. for 2 h. The reaction mixture was concentrated under vacuum then dissolved in DCM (10 mL), washed with water (10 mL) and sat. NH₄Cl (10 mL), dried over MgSO₄, filtered, and concentrated under vacuum. The product was purified by column chromatography (silica, 0:100→60:40 EtOAc:heptane) to afford 2-amino-6-(spiro[2.5]octan-7-ylmethyl)-4-(trifluoromethyl)-7H-pyrrolo[3,4-d]pyrimidin-5-one (192 mg, 50% yield) as a white solid. ¹H NMR (500 MHz, DMSO-d₆) δ 7.91-7.88 (m, 2H), 4.36 (d, J=4.1 Hz, 2H), 3.30-3.27 (m, 2H), 1.88-1.80 (m, 1H), 1.68-1.64 (m, 2H), 1.61-1.56 (m, 1H), 1.39-1.32 (m, 2H), 0.99-0.83 (m, 3H), 0.26-0.23 (m, 2H), 0.21-0.14 (m, 2H). ¹³C NMR (100 MHz, DMSO-d₆) δ 176.3, 163.7, 162.4, 150.7 (q, ²J_{CF}=37.5 Hz), 120.4, 119.9 (q, ¹J_{CF}=298.1), 109.1, 51.0, 47.3, 35.3, 35.0, 29.7, 24.2, 18.2, 11.8, 11.7. ¹⁹F NMR (470 MHz, DMSO-d₆) δ −66.09. HRMS (ESI) calcd. for [M+H]⁺ C₁₆H₂₀F₃N₄O 341.1589, found 341.1583.

### General scheme for the synthesis of 2-amino-4-(trifluoromethyl)-6-(((1R,3S)-3-(trifluoromethyl)cyclohexyl)methyl)-6,7-dihydro-5H-pyrrolo[3,4-d]pyrimidin-5-one (Compound 1), 2-amino-4-(trifluoromethyl)-6-(((1R,3R)-3-(trifluoromethyl)cyclohexyl)methyl)-6,7-dihydro-5H-pyrrolo[3,4-d]pyrimidin-5-one (Compound 5), 2-amino-4-(trifluoromethyl)-6-(((1S,3R)-3-(trifluoromethyl)cyclohexyl)methyl)-6,7-dihydro-5H-pyrrolo[3,4-d]pyrimidin-5-one (Compound 6), 2-amino-4-(trifluoromethyl)-6-(((1S,3S)-3-(trifluoromethyl)cyclohexyl)methyl)-6,7-dihydro-5H-pyrrolo[3,4-d]pyrimidin-5-one (Compound 7)

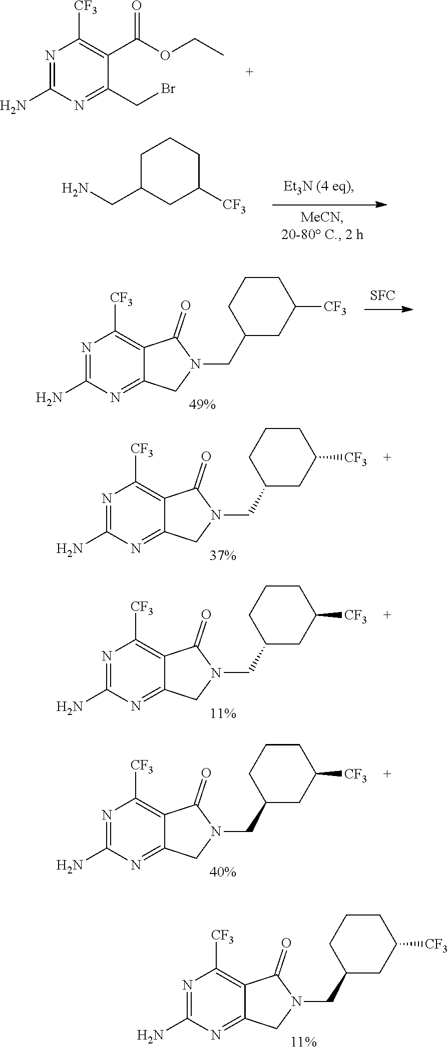

### 2-amino-4-(trifluoromethyl)-6-((3-(trifluoromethyl)cyclohexyl)methyl)-6,7-dihydro-5H-pyrrolo[3,4-d]pyrimidin-5-one (Intermediate 16)

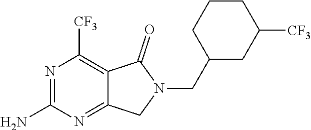 [SMILES: Nc1nc2c(c(C(F)(F)F)n1)C(=O)N(CC1CCCC(C(F)(F)F)C1)C2]

To a solution of 3-(trifluoromethyl)cyclohexyl)methanamine (144 mg, 0.8 mmol) and Et₃N (0.2 mL, 1.6 mmol) in MeCN (1 mL) was added ethyl 2-amino-4-(bromomethyl)-6-(trifluoromethyl)pyrimidine-5-carboxylate (130 mg, 0.4 mmol). The reaction mixture was stirred at 15° C. for 1 h, then heated to 80° C. for 1 h. The reaction mixture was concentrated under vacuum then dissolved in DCM (10 mL), washed with water (3×10 mL) and sat. NH₄Cl (10 mL). The product was purified by column chromatography (SiO₂, 5:95→45:55 EtOAc:heptane) to afford 2-amino-4-(trifluoromethyl)-6-((3-(trifluoromethyl)cyclohexyl)methyl)-6,7-dihydro-5H-pyrrolo[3,4-d]pyrimidin-5-one (75 mg, 49% yield) as a white solid. ¹H NMR (400 MHz, DMSO-d₆) δ 7.98-7.91 (m, 2H), 4.45-4.34 (m, 2H), 3.41-3.26 (m, 2H), 2.26-2.22 (m, 1H), 1.86-1.79 (m, 4H), 1.65 (br. d, J=11.2 Hz, 1H), 1.30-1.10 (m, 2H), 0.97-0.88 (m, 2H). MS (ES+): m/z (%) 383.2 (100) [M+H]⁺.

### 2-amino-4-(trifluoromethyl)-6-(((1R,3S)-3-(trifluoromethyl)cyclohexyl)methyl)-6,7-dihydro-5H-pyrrolo[3,4-d]pyrimidin-5-one (Compound 1)

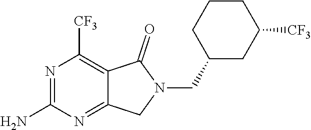 [SMILES: Nc1nc2c(c(C(F)(F)F)n1)C(=O)N(C[C@@H]1CCC[C@H](C(F)(F)F)C1)C2]

2-amino-4-(trifluoromethyl)-6-((3-(trifluoromethyl)cyclohexyl)methyl)-6,7-dihydro-5H-pyrrolo[3,4-d]pyrimidin-5-one was purified by SFC (column LuxCel2; solvent isopropanol, CO₂; isocratic mode (15% isopropanol)) then further purified by SFC (column LuxCel2; solvent methanol, CO₂; isocratic mode (10% methanol)) to afford 2-amino-4-(trifluoromethyl)-6-(((1R,3S)-3-(trifluoromethyl)cyclohexyl)methyl)-6,7-dihydro-5H-pyrrolo[3,4-d]pyrimidin-5-one (28 mg, 37% yield) as a white solid. ¹H NMR (400 MHz, DMSO-d₆) δ 8.11-7.77 (m, 2H), 4.48-4.32 (m, 2H), 3.41-3.23 (m, 2H), 2.29-2.16 (m, 1H), 1.86-1.73 (m, 4H), 1.65 (br. d, J=12.4 Hz, 1H), 1.35-1.09 (m, 2H), 0.99-0.77 (m, 2H). ¹³C NMR (125 MHz, DMSO-d₆) δ 176.4, 163.8, 162.6, 150.8 (q, ²J_{CF}=37.9 Hz), 128.0 (q, ¹J_{CF}=278.5 Hz), 119.9 (q, ¹J_{CF}=279.5 Hz), 117.9, 109.0, 51.2, 47.5, 34.8, 29.3, 28.6, 24.4, 23.6. ¹⁹F NMR (376 MHz, DMSO-d₆) δ −66.22, −72.48. HRMS (ESI) calcd. for [M+H]⁺ C₁₅H₁₇F₆N₄O, 383.1307, found 383.1305.

### 2-amino-4-(trifluoromethyl)-6-(((1R,3R)-3-(trifluoromethyl)cyclohexyl)methyl)-6,7-dihydro-5H-pyrrolo[3,4-d]pyrimidin-5-one (Compound 5)

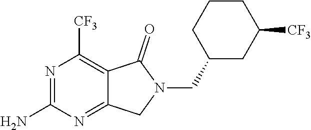 [SMILES: Nc1nc2c(c(C(F)(F)F)n1)C(=O)N(C[C@@H]1CCC[C@@H](C(F)(F)F)C1)C2]

2-amino-4-(trifluoromethyl)-6-((3-(trifluoromethyl)cyclohexyl)methyl)-6,7-dihydro-5H-pyrrolo[3,4-d]pyrimidin-5-one was purified by SFC (column LuxCel2; solvent isopropanol, CO₂; isocratic mode (15% isopropanol)) then further purified by SFC (column LuxCel2; solvent methanol, CO₂; isocratic mode (10% methanol)) to afford 2-amino-4-(trifluoromethyl)-6-(((1R,3R)-3-(trifluoromethyl)cyclohexyl)methyl)-6,7-dihydro-5H-pyrrolo[3,4-d]pyrimidin-5-one (8 mg, 11% yield) as a white solid. ¹H NMR (500 MHz, DMSO-d₆) δ 7.96-7.86 (m, 2H), 4.38 (d, J=7.3 Hz, 2H), 3.59-3.54 (m, 1H), 3.41-3.37 (m, 1H), 2.23-2.21 (m, 1H), 1.81-1.79 (m, 1H), 1.58-1.55 (m, 3H), 1.50-1.41 (m, 3H), 1.35-1.27 (m, 2H). HRMS (ESI) calcd. for [M+H]⁺ C₁₅H₁₇F₆N₄O, 383.1314, found 383.1339.

### 2-amino-4-(trifluoromethyl)-6-(((1S,3R)-3-(trifluoromethyl)cyclohexyl)methyl)-6,7-dihydro-5H-pyrrolo[3,4-d]pyrimidin-5-one (Compound 6)

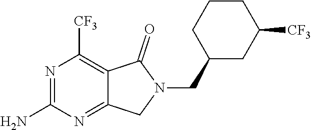 [SMILES: Nc1nc2c(c(C(F)(F)F)n1)C(=O)N(C[C@H]1CCC[C@@H](C(F)(F)F)C1)C2]

2-amino-4-(trifluoromethyl)-6-((3-(trifluoromethyl)cyclohexyl)methyl)-6,7-dihydro-5H-pyrrolo[3,4-d]pyrimidin-5-one was purified by SFC (column LuxCel2; solvent isopropanol, CO2; isocratic mode (15% isopropanol)) then further purified by SFC (column LuxCel2; solvent methanol, CO2; isocratic mode (10% methanol)) to afford 2-amino-4-(trifluoromethyl)-6-(((1 S,3R)-3-(trifluoromethyl)cyclohexyl)methyl)-6,7-dihydro-5H-pyrrolo[3,4-d]pyrimidin-5-one (30 mg, 40% yield) as a white solid. ¹H NMR (500 MHz, DMSO-d₆) δ 7.92-7.89 (m, 2H), 4.44-4.34 (m, 2H), 3.38-3.31 (m, 2H), 2.28-2.22 (m, 1H), 1.84-1.78 (m, 4H), 1.66 (d, J=12.4 Hz, 1H), 1.32-1.25 (m, 1H), 1.20-1.13 (m, 1H), 0.97-0.88 (m, 2H). ¹³C NMR (101 MHz, DMSO-d₆) δ 176.3, 163.8, 162.5, 150.8 (q, ²J_{CF}=37.0 Hz), 127.9 (q, ¹J_{CF}=283.1 Hz), 119.9 (q, ¹J_{CF}=260.9 Hz), 118.6, 109.0, 51.2, 47.5, 34.8, 29.3, 28.6, 24.3, 23.5. HRMS (ESI) calcd. for [M+H]⁺ C₁₅H₁₇F₆N₄O, 383.1267, found 383.1322.

### 2-amino-4-(trifluoromethyl)-6-(((1S,3S)-3-(trifluoromethyl)cyclohexyl)methyl)-6,7-dihydro-5H-pyrrolo[3,4-d]pyrimidin-5-one (Compound 7)

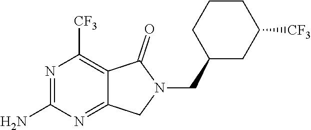 [SMILES: Nc1nc2c(c(C(F)(F)F)n1)C(=O)N(C[C@H]1CCC[C@H](C(F)(F)F)C1)C2]

2-amino-4-(trifluoromethyl)-6-((3-(trifluoromethyl)cyclohexyl)methyl)-6,7-dihydro-5H-pyrrolo[3,4-d]pyrimidin-5-one was purified by SFC (column LuxCel2; solvent isopropanol, CO2; isocratic mode (15% isopropanol)) then further purified by SFC (column LuxCel2; solvent methanol, CO2; isocratic mode (10% methanol)) to afford 2-amino-4-(trifluoromethyl)-6-(((1 S,3S)-3-(trifluoromethyl)cyclohexyl)methyl)-6,7-dihydro-5H-pyrrolo[3,4-d]pyrimidin-5-one (8 mg, 11% yield) as a white solid. ¹H NMR (500 MHz, DMSO-d₆) δ 7.95-7.88 (m, 2H), 4.42-4.34 (m, 2H), 3.59-3.54 (m, 1H), 3.41-3.37 (m, 1H), 2.23-2.21 (m, 1H), 1.81-1.79 (m, 1H), 1.58-1.55 (m, 3H), 1.49-1.42 (m, 3H), 1.35-1.28 (m, 2H). HRMS (ESI) calcd. for [M+H]⁺ C₁₅H₁₇F₆N₄O, 383.1291, found 383.1324.

### Biological Data and Experimental Results

For the avoidance of doubt, where activity data is presented as an pIC50 value this is not the same as an IC50 value. Both pIC₅₀ and IC₅₀ are terms of the art and it will be readily appreciated by the skilled person that compounds which have an IC₅₀ value of less than about 10 μM will have a corresponding pIC₅₀ value of greater than about 5.

Generally speaking, an IC₅₀ value of 1 μM is equivalent to a pIC₅₀ value of 6, and an IC₅₀ value of 100 nM is equivalent to a pIC₅₀ value of 7.

### Protein Expression and Purification Method for Recombinant CpKRS

The protein expression and purification method for recombinant CpKRS was described in Baragana et aL, PNAS, 2019, 116 (4), 7015-7020 and will be summarised below.

The C. parvum KRS had 45 residues removed at the N-terminus. All amplicons were cloned into the pAVA0421 expression vector using the ligation independent cloning (LIC) method. Recombinant plasmids were transformed into Rosetta BL21(DE3) competent cells and plated on LB agar plates with ampicillin, carbenicillin, and chloramphenicol selection. Clones were expressed using previously reported autoinduction methods and scaled to 2-litre cultures in a shake flasks. Cell pellets were harvested and lysed with 100 mM HEPES/150 mM NaCl/5% Glycerol/20 mM imidazole/0.5 mM TCEP pH 7.5/DNase/Complete inhibitor tablets) using a Cell Disrupter (Constant Systems) at 30 KPSI and centrifuged at 40,000 g for 20 min remove cell debris. The supernatant was loaded onto a 5 ml HiTrap Ni HP column that had been equilibrated with Buffer A (100 mM HEPES/150 mM NaCl/5% Glycerol/20 mM imidazole/0.5 mM TCEP pH 7.5) at 5 ml/min using an AKTA Pure system. Once loaded the column was washed with 10 column volumes of buffer A. A 5% step of Buffer B (100 mM HEPES/150 mM NaCl/5% Glycerol/500 mM imidazole/0.5 mM TCEP pH 7.5) was then used to wash off His-rich contaminating proteins. A linear gradient of 5-50% B was used to elute the protein. Approximately 287 mg of protein was present in the fractions containing the CpKRS protein. The sample was then concentrated to approximately 30 ml, passed through a 0.2 μm filter, before loading onto a XK26/60 Superdex 200 column using an AKTA Pure system at 4° C. at 2 ml/min 10 ml at a time. The eluted protein was pooled then frozen at −80° C. The concentration of the protein was 1.3 mg/ml. Sometimes the protein was cleaved with PreScission protease overnight, a second Ni column performed in flow through mode to removed uncleaved protein. GST beads were then added to remove the PreScission Protease. The protein was then gel filtered as descripted above.

### Protein Expression and Purification Method for Recombinant HsKRS

The gene coding for Human KRS, codon optimized for expression in E. coli, was obtained from Genscript and cloned into a His PP pET15b vector using Nde1 and Xho1 restriction sites. The resulting vector was used to transform BL21 (DE3) competent cells for protein expression. A 120 ml overnight culture was set up and grown at 37° C. 200 rpm for 16 h and used the next day to inoculate 6 litres of Autoinduction+AMP medium. This culture was grown at 37° C. for 4 h then 20° C. for 20 h. The cells were harvested by centrifugation at 3,500 g for 30 min then stored at −20° C. The resulting cell pellet was 50 g. Lysis buffer (150 ml, 25 mM HEPES/500 mM NaCl/20 mM imidazole/2 mM DTT/10% glycerol pH 7.5/protease inhibitor tablets/DNAase) was added and the pellets for defrosted at room temperature for approximately 10 min. The slurry was then passed through a Cell Disrupter (Constant Systems) set at KPSI to lyse the cells. The sample was then centrifuged at 40,000 g for 20 min. The supernatant was then filtered using syringe filters to 0.45 μm. The supernatant was loaded onto a 5 ml HiTrap Ni HP column that had been equilibrated with BufferA (25 mM HEPES/500 mM NaCl/20 mM Imidazole/2 mM DTT/10% Glycerol pH 7.5) at 5 ml/min using an AKTA Pure system. Once loaded the column was washed with 10 column volumes buffer A. A 5% step of Buffer B (25 mM HEPES/500 mM NaCl/500 mM Imidazole/2 mM DTT/10% Glycerol pH 7.5) was then used to wash off His rich contaminating proteins. A gradient of 5-50% B was used to elute the protein. Approximately 55 mg of protein was present in the fractions containing the HsKRS protein. The sample was then passed through a 0.2 μm filter, before loading onto a XK26/60 Superdex 200 column equilibrated with Buffer C (25 mM HEPES/150 mM NaCl/2 mM DTT/10% glycerol pH 7.0) using an AKTA Pure system at 4° C. at 2 ml/min. The sample was then dialyzed into 25 mM HEPES/500 mM NaCl//2 mM DTT/10% glycerol/0.25% azide pH 7.0. The protein was concentrated to 1 mg/ml and frozen at −80° C. Approximately 23 mg of protein was produced.

Assay Method for Measurement of In Vitro Inhibition of Cryptosporidium lysyl tRNA Synthetase (KRS)

Potency of compounds was determined in 10 points dose-response inhibition curves. Compounds were transferred into 384-well clear bottom plates (Greiner Bio-One, 781101) using an Echo 550 acoustic dispenser (Labcyte). Using an automatic liquid dispenser, 50 μL reaction mixes were prepared by adding 25 μL of substrate mix into all wells followed by the addition of 25 μL enzyme mix into compounds and no inhibition control wells (final reaction: 100 mM HEPES, pH 7.4, 20 mM MgCl₂, 1 mM DTT, 500 μM ATP, 1.2 mM L-Lysine, 300 nM CpKRS and 0.5 U/mL Pyrophosphatase). Complete inhibition control reactions were performed in the absence of enzyme and the plates were incubated at room temperature for 8 h. Reactions were stopped by the addition of Biomol green (50 μL: Enzo Life Sciences) with the amount of free phosphate detected by measuring absorbance (650 nm) after 20 min further incubation (BMG Pherastar plate reader). Counter screen assays to eliminate direct inhibition of pyrophosphatase were performed using 0.5 U/mL pyrophosphatase and 3 μM pyrophosphate as substrate in the same assay buffer (100 mM HEPES, pH 7.4, 20 mM MgCl2, 1 mM DTT) for 8 hours at room temperature. Biomol green was added and incubated for 20 min before detection as above. Samples were run in duplicate and data was processed and analysed using ActivityBase (IDBS).

Assay Method for Measurement of In Vitro Inhibition of Human lysyl tRNA Synthetase (KRS)

Potency of compounds was determined in 10 points dose-response inhibition curves. Compounds were transferred into 384-well clear bottom plates (Greiner Bio-One, 781101) using an Echo 550 acoustic dispenser (Labcyte). Using an automatic liquid dispenser, 50 μL reaction mixes were prepared by adding 25 μL of substrate mix into all wells followed by the addition of 25 μL enzyme mix into all wells (final reaction: 30 mM Tris-HCl, pH 8.0, 40 mM MgCl2, 140 mM NaCl, 30 mM KCl, 0.01% Brij-35, 1 mM DTT, 3.5 μM ATP, 6 μM L-Lysine, 200 nM CpKRS and 0.5 U/mL Pyrophosphatase). Complete inhibition control reactions were performed in the presence of 100 μM of control compoundand the plates were incubated at room temperature for 5 h. Reactions were stopped by the addition of Biomol green (50 μL: Enzo Life Sciences) with the amount of free phosphate detected by measuring absorbance (650 nm) after 20 min further incubation (BMG Pherastar plate reader). Counter screen assays to eliminate direct inhibition of pyrophsphatase were performed using 0.5 U/mL pyrophosphatase and 3 μM pyrophosphate as substrate in the same assay buffer (30 mM Tris-HCl, pH 8.0, 40 mM MgCl₂, 140 mM NaCl, 30 mM KCl, 0.01% Brij-35, 1 mM DTT) for 5 hours at room temperature. Biomol green was added and incubated for 20 min before detection as above. Samples were run in duplicate and data was processed and analysed using ActivityBase (IDBS).

### Assay Method for Measurement of In Vitro Inhibition of Cryptosporidium

The in vitro measurement of ECso, the effective concentration of compound causing 50% parasite death versus Crytosporidium exhibited by compounds of Formula I, I-A, I-B, I-C, I-D, I-E, I-F and/or I-G, has been carried out. These experiments were run in accordance with the method of Besssoff et. al. Antimicrob. Agents Chemother. 2013, 57:1804-1814. Results from these experiments are provided in the Experimental Results hereinafter.

### Assay Method for Measurement of In Vitro Inhibition of HepG2 Cells (Cytotoxicity)

The in vitro measurement of ECso, the effective concentration of compound causing 50% parasite death versus Hep G2 (Human Caucasian hepatocyte carcinoma, HPACC cat. no. 85011430) exhibited by compounds of Formula I, I-A, I-B, I-C, I-D, I-E, I-F and/or I-G, has been carried out. These experiments were run in accordance with the method of Mersch-Sundermann V et. al. Toxicology 2004, 198:329-340

Results from these experiments are provided in the Experimental Results hereinafter.

### Measurement of Anti-Cryptosporidium In Vivo Efficacy

The in vivo measurement of the potential efficacy of compounds of formula (I) in a cryptosporidium mouse model has been carried out following the method described in Antimicrob. Agents Ch., 2018, 62, 4, e01505-17, and which is summarised below.

The cryptosporidiosis NOD SCID gamma mouse model was run as follows: Male NOD SCID gamma mice (NOD.Cg-Prkdc^{scid} II2rg^{tm1Wjt}/SzJ, Jackson Labs) were infected approximately 2 weeks post weaning with 10\hat{ }5 C. parvum Iowa strain oocysts by oral gavage. Four mice were used per experimental group. This establishes a chronic, asymptomatic infection of the small intestine, cecum, and biliary tree. Fecal parasite shedding is monitored by quantitative PCR to amplify C. parvum DNA. One week after infection (which is the timing with which shedding becomes uniformly detected), compounds are dosed by oral gavage. Treatment continued for seven days total. The number of oocysts per milligram of stool was measured for each mouse on day 8 (1 day after completion of the last dose) and day 15 15 (7 days after the completion of the last dose). Average oocyst concentrations were calculated for each group of mice. Log reduction is the average reduction in oocyst shedding compared to untreated vehicle.

These experiments were run in accordance with the method of C. D. Huston et al, Antimicrob. Agents Ch., 2018, 62, 4, e01505-17. The in vivo results are provided in Table 3.

Results from these experiments are provided in the Experimental Results hereinafter.

### Biological Activity

Compounds of Formula I, I-A, I-B, I-C, I-D, I-E, I-F and/or I-G have demonstrated Crytosporidium inhibitory activity. The Cryptosporidium parvum pEC₅₀ data for some of the Exemplary compounds herein from these in vitro Crytosporidium inhibition assay tests are provided in Table 2.

Compounds of Formula I, I-A, I-B, I-C, I-D, I-E, I-F and/or I-G have demonstrated cellular selectivity. The Hep G2 pEC₅₀ data for some Exemplary compounds herein from these Hep G2 In-vitro cytotoxicity assays are provided in Table 2.

Compounds of Formula I, I-A, I-B, I-C, I-D, I-E, I-F and/or I-G have demonstrated Cp KRS inhibitory activity. The Cp KRS pIC₅₀ data for some of the Exemplary compounds herein from these Cryptosporidium parvum lysyl t-RNA synthetase (Cp KRS) inhibition assay tests are provided in Table 2.

TABLE 2
Biology results

| | | C. parvum | | C. parvum | Hs HepG2 |
|---|---|---|---|---|---|
| Compound | Structure | KRS pIC₅₀ | Hs KRS pIC₅₀ | PEC₅₀ | PEC₅₀ |
| 1 | 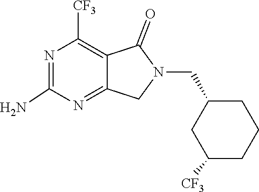 [SMILES: Nc1nc2c(c(C(F)(F)F)n1)C(=O)N(C[C@@H]1CCC[C@H](C(F)(F)F)C1)C2] | 6.1 | 4.7 | 7.5 | 4.6 |
| 2 | 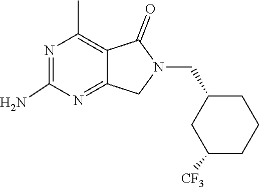 [SMILES: Cc1nc(N)nc2c1C(=O)N(C[C@@H]1CCC[C@H](C(F)(F)F)C1)C2] | 6.1 | 4.7 | 7.7 | 4.9 |
| 3 | 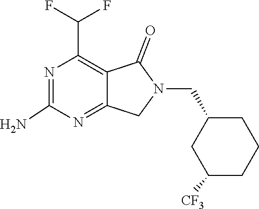 [SMILES: Nc1nc2c(c(C(F)F)n1)C(=O)N(C[C@@H]1CCC[C@H](C(F)(F)F)C1)C2] | 6.0 | <4 | 7.6 | 4.6 |
| 4 | 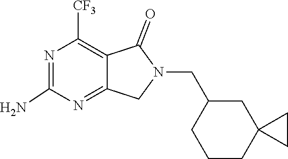 [SMILES: Nc1nc2c(c(C(F)(F)F)n1)C(=O)N(CC1CCCC3(CC3)C1)C2] | 5.6 | <4 | 6.8 | 4.7 |
| 5 | 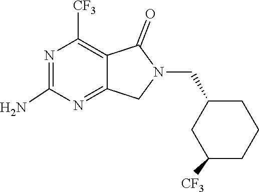 [SMILES: Nc1nc2c(c(C(F)(F)F)n1)C(=O)N(C[C@@H]1CCC[C@@H](C(F)(F)F)C1)C2] | 5.8 | 4.7 | 7.3 | 4.8 |
| 6 | 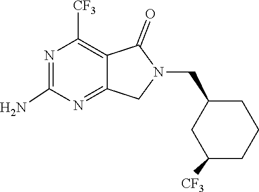 [SMILES: Nc1nc2c(c(C(F)(F)F)n1)C(=O)N(C[C@H]1CCC[C@@H](C(F)(F)F)C1)C2] | <4.3 | <4 | N.D. | <4 |
| 7 | 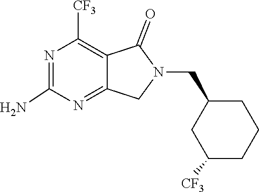 [SMILES: Nc1nc2c(c(C(F)(F)F)n1)C(=O)N(C[C@H]1CCC[C@H](C(F)(F)F)C1)C2] | 4.8 | 4.2 | N.D | 4.1 |

TABLE 3
In vivo efficacy result for average reduction in oocyst shedding compared to untreated vehicle

| | | | | | | Log | Log |
|---|---|---|---|---|---|---|---|
| | | Dose | Regimen | | | Reduction | Reduction |
| Compound | Structure | (mg/kg) | Route | Freq | Days | (day 8) | (day 15) |
| 1 | 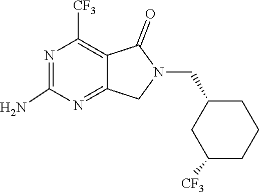 [SMILES: Nc1nc2c(c(C(F)(F)F)n1)C(=O)N(C[C@@H]1CCC[C@H](C(F)(F)F)C1)C2] | 10 20 30 40 50 | P.O. P.O. P.O. P.O. P.O. | B.I.D. B.I.D. B.I.D. B.I.D. B.I.D. | 7 7 7 7 7 | 3.0 3.1 2.9 2.6 3.4 | 1.0 0.7 2.2 2.9 3.5 |
| 2 | 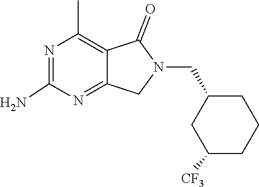 [SMILES: Cc1nc(N)nc2c1C(=O)N(C[C@@H]1CCC[C@H](C(F)(F)F)C1)C2] | 10 30 | P.O. P.O. | B.I.D. B.I.D. | 7 7 | 1.8 2.7 | 0.8 2.4 |
| 3 | 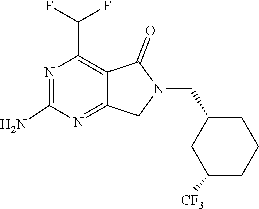 [SMILES: Nc1nc2c(c(C(F)F)n1)C(=O)N(C[C@@H]1CCC[C@H](C(F)(F)F)C1)C2] | 10 30 | P.O. P.O. | B.I.D. B.I.D. | 7 7 | 2.4 2.8 | 1.0 1.6 |
| 4 | 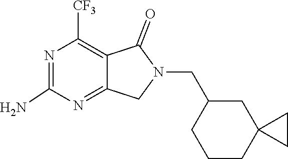 [SMILES: Nc1nc2c(c(C(F)(F)F)n1)C(=O)N(CC1CCCC3(CC3)C1)C2] | 10 50 | P.O. P.O. | B.I.D. B.I.D. | 7 7 | 1.3 3.4 | 1.0 2.5 |

1. A compound of Formula I: 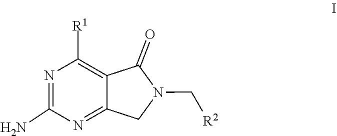
wherein R¹ is selected from: —CH₃, —CF₂H or —CF₃; and
wherein R² is selected from: 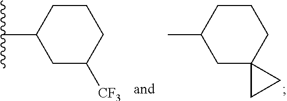
or a veterinarily or pharmaceutically acceptable, salt, hydrate, solvate, isomer, prodrug or polymorph thereof.

2. A compound of Formula I according to claim 1, wherein R¹ is —CH₃ or wherein R¹ is —CF₃.

3. (canceled)

4. A compound of Formula I according to claim 1, wherein R² is

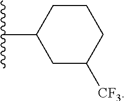 [SMILES: *C1CCCC(C(F)(F)F)C1]

5. A compound of Formula I according to claim 1, wherein the compound of Formula I is a compound of Formula I-A: 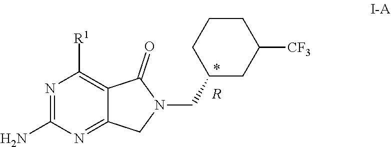
wherein R¹ is selected from: —CH₃, —CF₂H or —CF₃;
or a veterinarily or pharmaceutically acceptable, salt, hydrate, solvate, isomer, prodrug or polymorph thereof.

6. A compound of Formula I-A according to claim 5, wherein the compound of Formula I-A is a compound of Formula I-B: 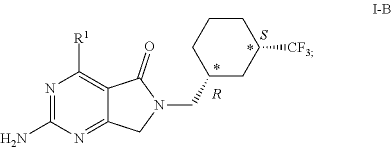
and
wherein R¹ is selected from: —CH₃, —CF₂H or —CF₃;
or a veterinarily or pharmaceutically acceptable, salt, hydrate, solvate, isomer, prodrug or polymorph thereof.

7. A compound of Formula I-A according to claim 5, wherein the compound of Formula I-A is a compound of Formula I-C: 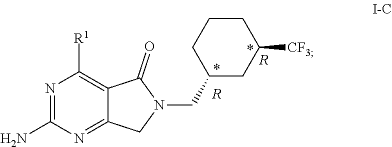
and
wherein R¹ is selected from: —CH₃, —CF₂H or —CF₃;
or a veterinarily or pharmaceutically acceptable, salt, hydrate, solvate, isomer, prodrug or polymorph thereof.

8. A compound of Formula I according to claim 1, wherein the compound of Formula I is a compound of Formula I-D: 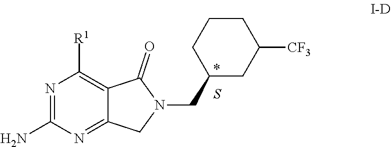
wherein R¹ is selected from: —CH₃, —CF₂H or —CF₃;
or a veterinarily or pharmaceutically acceptable, salt, hydrate, solvate, isomer, prodrug or polymorph thereof.

9. A compound of Formula I-D according to claim 8, wherein the compound of Formula I-D is a compound of Formula I-E: 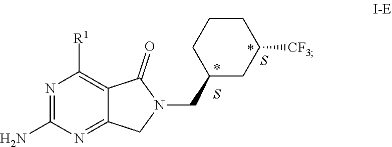
and
wherein R¹ is selected from: —CH₃, —CF₂H or —CF₃;
or a veterinarily or pharmaceutically acceptable, salt, hydrate, solvate, isomer, prodrug or polymorph thereof.

10. A compound of Formula I-D according to claim 8, wherein the compound of Formula I-D is a compound of Formula I-F: 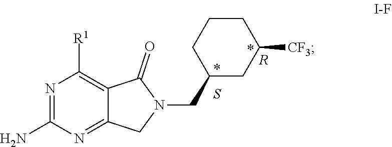
and
wherein R¹ is selected from: —CH₃, —CF₂H or —CF₃;
or a veterinarily or pharmaceutically acceptable, salt, hydrate, solvate, isomer, prodrug or polymorph thereof.

11. (canceled)

12. (canceled)

13. A compound of Formula I according to claim 1, wherein the compound of Formula I is a compound of Formula I-G: 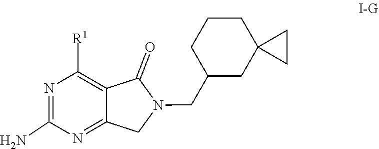
wherein R¹ is selected from: —CH₃, —CF₂H or —CF₃;
or a veterinarily or pharmaceutically acceptable, salt, hydrate, solvate, isomer, prodrug or polymorph thereof.

14. A compound of Formula I-G according to claim 13,
wherein R¹ is —CH₃ or wherein R¹ is —CF₃.

15. (canceled)

16. A compound of Formula I according to claim 1, wherein the compound is selected from: 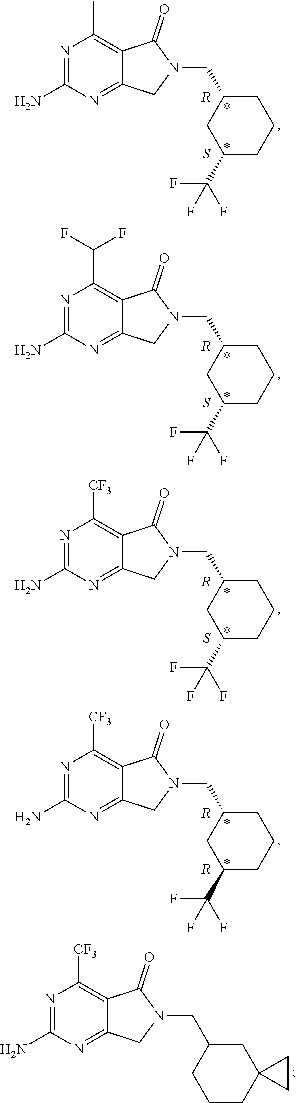
or a veterinarily or pharmaceutically acceptable, salt, hydrate, solvate, isomer, prodrug or polymorph thereof.

17. A compound according to claim 1 having a pEC₅₀ for Crytosporidium parvum of 6 or more, 6.5 or more, 7 or more, 7.3 or more, or 7.7 or more.

18. A compound according to claim 1 having a pIC₅₀ for Crytosporidium parvum lysyl t-RNA synthetase (Cp KRS) of 5 or more, 5.5 or more, or 6 or more.

19. A pharmaceutical composition comprising a compound according to claim 1, for use in medicine or for veterinary use.

20. A method of treatment of an infectious disease comprising administering to a subject in need a compound of formula I according to claim 1.

21. A method of treatment or prevention of a disease caused by Cryptosporidium, optionally wherein the disease is caused by a Cryptosporidium species selected from: Cryptosporidium andersoni, Cryptosporidium baileyi, Cryptosporidium bovis, Cryptosporidium canis, Cryptosporidium chipmunk, Cryptosporidium cuniculus, Cryptosporidium ducismarci, Cryptosporidium felis, Cryptosporidium fayeri, Cryptosporidium galli, Cryptosporidium meleagridis, Cryptosporidium muris, Cryptosporidium monari, Cryptosporidium suis, Cryptosporidium scrofarum, Cryptosporidium tyzzeri, Cryptosporidium ubiquitum, Cryptosporidium viatorum, Cryptosporidium nasorum, Cryptosporidium parvum, Cryptosporidium hominis, Cryptosporidium saurophilum, Cryptosporidium serpentis, and Cryptosporidium wrairi, wherein the method comprises administering to a human subject or mammal in need thereof a therapeutically effective amount of a compound according to claim 1 or a pharmaceutically acceptable salt, solvate, hydrate, isomer, prodrug, or polymorph thereof.

22. A pharmaceutical composition comprising a a pharmaceutically acceptable salt, solvate, hydrate, isomer, prodrug, or polymorph thereof.
